(19) United States
(12) Patent Application Publication
(10) Pub. No.: US 2023/0381226 A1
(43) Pub. Date: Nov. 30, 2023

(54) SURFACE-ENGINEERED EXTRACELLULAR VESICLES AND THERAPEUTIC USES THEREOF

(71) Applicants: SHIFTBIO INC., Seoul (KR); Korea Institute of Science and Technology, Seoul (KR)

(72) Inventors: In San KIM, Seoul (KR); Seungyoon PARK, Seoul (KR); Gi-Hoon NAM, Seoul (KR); Inkyu LEE, Seoul (KR)

(21) Appl. No.: 18/322,723

(22) Filed: May 24, 2023

Related U.S. Application Data
(60) Provisional application No. 63/345,040, filed on May 24, 2022.

(51) Int. Cl. A61K 35/12; C12N 15/85; C12N 5/00

(52) U.S. Cl. CPC A61K 35/12; C12N 15/85; C12N 5/0006; C12N 2800/107

(57) ABSTRACT

The present invention provides surface-engineered extracelluar vesicles, compositions comprising the surface-engineered extracelluar vesicles, methods for preparing the surface-engineered extracelluar vesicles, and methods for using the surface-engineered extracelluar vesicles or the compositions.

### CROSS-REFERENCE TO RELATED APPLICATION(S)

This application claims priority from 63/345,040 filed on May 24, 2022, which is incorporated herein by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on May 24, 2023, is named “Seq_SFT-P30002.xml” and is 143,044 bytes in size. The sequence listing contained in this .XML file is part of the specification and is hereby incorporated by reference herein in its entirety.

### TECHNICAL FIELD

The present invention generally relates to surface-engineered extracellular vesicles, compositions comprising the surface-engineered extracellular vesicles, methods for preparing the surface-engineered extracellular vesicles, and methods for using the surface-engineered extracellular vesicles or the compositions.

### BACKGROUND

A considerable amount of effort has been made to use exosomes to deliver to desired target cells for therapeutic purposes a variety of therapeutic molecules, examples of which include therapeutic protein, membrane protein, protein reporters, enzymes, antibody fragments, cytokines, tumor necrosis factor superfamily (TNFSF) ligands, RNA binding proteins, Cas9, and vaccine antigens. Exosomes having therapeutic molecules on the surface thereof were proposed. Scaffolds that can display therapeutic molecules on the surface of the exosomes were proposed. Prostaglandin F2 receptor regulatory protein (PTGFRN) was proposed as a scaffold for displaying on the surface of exosomes various therapeutic molecules. There is, however, still a need for a new scaffold that can display therapeutic molecules on the surface of exosomes in a better way.

### SUMMARY

An aspect of the present invention provides a DNA construct comprising a DNA sequence encoding a scaffold peptide, wherein the amino sequence of the scaffold peptide includes a sequence represented by G-a-S-b-X1-c-X2 (extracellular vesicle sorting motif, ESM), in which X1 represents G, A, S or T; X2 represents G or S; a represents 3-4 amino acids; b represents 2-3 amino acids; and c represents 6-7 amino acids; G represents glycine; S represents serine; A represents alanine; and T represents threonine.

In some embodiments, the sequence G-a-S-b-X1-c-X2 may have 15-17 amino acids. In some embodiments, the scaffold peptide may have 22-57 amino acids. In some embodiments, the amino acids a, b, and c may include V, G, L, I, A, T, S, C, F, W, Y, and P, in which V represents valine, G represents glycine, L represents leucine, I represents isoleucine, A represents alanine, T represents threonine, S represents serine, C represents cysteine, F represents phenylalanine, W represents tryptophan, Y represents tyrosine, and P represents proline. In some embodiments, a may represent 3-4 amino acids selected from the group consisting of V, G, L, I, T and A, in which V represents valine, G represents glycine, L represents leucine, I represents isoleucine, T represents threonine and A represents alanine. In some embodiments, a may represent VGL, IGL, VGLT, IGLT, VGLA or IGLA. In some embodiments, b may represent 2-3 amino acids selected from the group consisting of V, I, A and T, in which V represents valine, I represents isoleucine, A represents alanine and T represents threonine. In some embodiments, b may represent VI, AV, TVI or AVI. In some embodiments, c may represent 6-7 amino acids selected from the group consisting of L, S, C and I, in which L represents leucine, S represents serine, C represents cysteine, and I represents isoleucine. In some embodiments, c may represent LLSCLI or ILLSCLI. In some embodiments, the sequence G-a-S-b-X1-c-X2 may be one of the amino acid sequence as set forth in ESM SEQ ID NOS: 1-100. In some embodiments, the scaffold peptide may further comprise KYPLLI at the N-terminal of the sequence G-a-S-b-X1-c-X2, in which K represents lysine, Y represents tyrosine, P represents proline, L represents leucine, and I represents isoleucine. In some embodiments, the scaffold peptide may further comprise DVLNAFKYPLLI at the N-terminal of the sequence G-a-S-b-X1-c-X2, in which D represents aspartic acid, V represents valine, L represents leucine, N represents asparagine, A represents alanine, F represents phenylalanine, K represents lysine, Y represents tyrosine, P represents proline, L represents leucine, and I represents isoleucine. In some embodiments, the scaffold peptide may further comprise YCSS at the C-terminal of the sequence G-a-S-b-X1-c-X2, in which Y represents tyrosine, C represents cysteine, and S represents serine. In some embodiments, the scaffold peptide may further comprise YCSSHWCCKKEVQETRRERRRLMSMEMD at the C-terminal of the sequence G-a-S-b-X1-c-X2, in which Y represents tyrosine, C represents cysteine, S represents serine, H represents histidine, W represents tryptophan, K represents lysine, E represents glutamic acid, V represents valine, Q represents glutamine, T represents threonine, R represents arginine, L represents leucine, M represents methionine, and D represents aspartic acid. In some embodiments, the scaffold peptide may further comprise YCSSHWC at the C-terminal of the sequence G-a-S-b-X1-c-X2, in which Y represents tyrosine, C represents cysteine, S represents serine, H represents histidine, and W represents tryptophan. In some embodiments, the DNA construct may further comprising a DNA sequence encoding an amino acid sequence of a target protein. In some embodiments, the target protein may be a therapeutic protein. In some embodiments, the scaffold peptide may display the target protein at a desired position of an extracellular vesicle. In some embodiments, the desired position may be an inner surface or an outer surface of the extracellular vesicle.

A further aspect of the present invention provides a vector comprising the above-described DNA construct. A non-limiting example of the vector is an expression plasmid including a DNA sequence encoding the scaffold peptide.

A still further aspect of the present invention provides a host cell comprising the above-described vector. Non-limiting examples of the host cell may include an HEK293 cell, a Chinese hamster ovary (CHO) cell, a mesenchymal stem cell (MSC), and cells derived from the HEK293 cell, CHO cell, or MSC. In addition, non-limiting examples of the host cell may include mast cells, immune cells, Natural killer cells, dendritic cells, macrophages, T lymphocytes, B lymphocytes, epithelial cells, human cardiac progenitor cells, adipose-derived stem cells, umbilical cord blood-derived mesenchymal stem cells, and bone marrow-mesenchymal stem cells.

A still yet further aspect of the present invention provides an extracellular vesicle (EV) isolated from the above-described host cell. In some embodiments, the scaffold peptide may be displayed at a desired position of the extracellular vesicle. For example, the scaffold peptide may be displayed on the inner surface of the extracellular vesicle, the outer surface of the extracellular vesicle, or both. In some embodiments, the extracellular vesicle may further comprise a target protein. In some embodiments, the scaffold peptide may be fused to the target protein. In some embodiments, the scaffold peptide may comprise an affinity tag having to a binding agent. In some embodiments, the extracellular vesicle may further comprise a targeting moiety. In some embodiments, the extracellular vesicle may further comprise a therapeutic substance.

When extracellular vesicles include the scaffold peptide of the present invention, the extracellular vesicles may have the scaffold peptide displayed on the surface(s) of the extracellular vesicles at a higher density, compared with when extracellular vesicles include a scaffold peptide different from the scaffold peptides of the present invention. Non-limiting examples of the scaffold peptide different from the scaffold peptide of the present invention may include a conventional extracellular vesicle protein, a fragment, or variant thereof, a fragment of the variant, and a variant of the fragment. When extracellular vesicles include the scaffold peptide of the present invention, the extracellular vesicles may include a higher amount of the target protein, compared with when extracellular vesicles include a scaffold peptide different from the scaffold peptides of the present invention.

A still yet further aspect of the present invention provides an extracellular vesicle comprising the scaffold peptide encoded by the above-described DNA construct. In some embodiments, the scaffold peptide may be displayed at a desired position of the extracellular vesicle. For example, the scaffold peptide may be displayed on the inner surface of the extracellular vesicle, the outer surface of the extracellular vesicle, or both.

A still yet another aspect of the present invention provides a pharmaceutical composition comprising the above-described extracellular vesicle. In some embodiments, the pharmaceutical composition may further comprise a pharmaceutically acceptable carrier.

A yet further aspect of the present invention provides a method for preventing, ameliorating, or treating disease, disorder, or condition associated with nervous, digestive, endocrine, skeletal, respiratory, integumentary, lymphatic, reproductive, muscular, excretory, or immune system, the method comprising administering to a subject in need a therapeutically effective amount of the above-described pharmaceutical composition. In some embodiments, the disease, disorder, or condition may be at least one selected from the group consisting of certain infectious or parasitic diseases, neoplasms, diseases of the blood or blood-forming organs, diseases of the immune system, endocrine, nutritional or metabolic diseases, mental, behavioral or neurodevelopmental disorders, sleep-wake disorders, diseases of the nervous system, diseases of the visual system, diseases of the ear or mastoid process, diseases of the circulatory system, diseases of the respiratory system, diseases of the digestive system, diseases of the skin, diseases of the musculoskeletal system or connective tissue, diseases of the genitourinary system, conditions related to sexual health, diseases of the obstetrics and gynecology, developmental anomalies, certain conditions originating in the perinatal period, symptoms, signs or clinical findings, not elsewhere classified, injury, poisoning or certain other consequences of external causes, external causes of morbidity or mortality.

A still yet further aspect of the present invention provides a method for preparing a surface-engineered extracellular vesicle for therapeutic use. Particularly, the above-described DNA construct and/or the above-described scaffold peptide is/are used to prepare a surface-engineered extracellular vesicle. In some embodiments, a target protein (e.g., a therapeutic protein) and the scaffold peptide may be conjugated to prepare a fusion protein, and the fusion protein may be to be displayed on the surface of an extracellular vesicle, thereby preparing a surface-engineered extracellular vesicle. Surface-engineered extracellular vesicles prepared by the methods using the above-described scaffold peptide in accordance with the present invention is better in many aspects than surface-engineered extracellular vesicles prepared by methods using some other scaffolds (e.g., PTGFRN). For example, a therapeutic protein of interest, a scaffold, or both are displayed on the surface of the extracellular vesicles prepared by the methods in accordance with the present invention at a higher density than those are displayed on the surface of the extracellular vesicles prepared by the methods using some other scaffolds (e.g., PTGFRN) are. Also, the surface-engineered extracellular vesicles prepared by the methods in accordance with the present invention exhibit higher therapeutic efficacy than the surface-engineered extracellular vesicles prepared by the methods using some other scaffolds (e.g., PTGFRN) does. In addition, the scaffold peptide of the present invention is shorter than some other scaffolds (e.g., PTGFRN). Also, therapeutic proteins are displayed on the surface of the extracellular vesicles prepared by the methods in accordance with the present invention more effectively than therapeutic proteins are displayed on the surface of the extracellular vesicles prepared by the methods using some other scaffolds (e.g., PTGFRN).

The above and other aspects and embodiments of the present invention will be discussed in more detail below.

### BRIEF DESCRIPTION OF THE DRAWINGS

The figures depict various embodiments of the present invention for purposes of illustration only. One skilled in the art will readily recognize from the following discussion that alternative embodiments of the structures and methods illustrated herein may be employed without departing from the principles of the invention described herein.

FIG. 1 illustrates the DNA constructs of some embodiments of the present invention, the signal-regulatory protein alpha (SIRPα) and CD81 protein expression of the constructs in extracellular vesicles, and the semi-quantitative analysis of SIRPα protein expression normalized by CD81 in extracellular vesicles;

FIG. 2 illustrates the DNA constructs of some embodiments of the present invention, the SIRPα and CD81 protein expression of the constructs in extracellular vesicles, the semi-quantitative analysis of SIRPα protein expression normalized by CD81 in extracellular vesicles, and the SIRPα and actin protein expression of the constructs in cell lysates;

FIG. 3 illustrates the DNA constructs of some embodiments of the present invention, the SIRPα and CD81 protein expression of the constructs in extracellular vesicles, the semi-quantitative analysis of SIRPα protein expression normalized by CD81 in extracellular vesicles, and the SIRPα and actin protein expression of the constructs in cell lysates;

FIG. 4 illustrates the DNA constructs of some embodiments of the present invention, the epidermal growth factor (EGF) and CD81 protein expression of the constructs in extracellular vesicles, and the semi-quantitative analysis of EGF protein expression normalized by CD81 in extracellular vesicles;

FIG. 5 illustrates the DNA constructs of some embodiments of the present invention, and the EGF protein expression of the constructs in extracellular vesicles or cell lysates;

FIG. 6 illustrates the DNA constructs of some embodiments of the present invention, and the EGF and CD81 protein expression of the constructs in extracellular vesicles or cell lysates;

FIG. 7 illustrates the DNA constructs of still yet other embodiments of the present invention;

FIG. 8 illustrates the SIRPα and CD81 protein expression of the DNA constructs based on FIG. 7 in extracellular vesicles;

FIG. 9 illustrates the relative SIRPα expression in normalized by CD81 extracellular vesicles of FIG. 8;

FIG. 10 illustrates the EGF and CD81 protein expression of the DNA constructs based on FIG. 7 in extracellular vesicles;

FIG. 11 illustrates the relative EGF expression in normalized by CD81 extracellular vesicles of FIG. 10.

FIG. 12 illustrates the DNA constructs of some embodiments of the present invention, and the SIRPα protein expression of the constructs in extracellular vesicles;

FIG. 13 illustrates the DNA constructs of some embodiments of the present invention, the SIRPα and actin protein expression of the constructs in cell lysates, and the SIRPα and CD81 protein expression of the constructs in extracellular vesicles;

FIG. 14 illustrates the relative SIRPα expression in extracellular vesicles of FIG. 13;

FIG. 15 illustrates HEK293 cells stably transduced with the K-SIRPα-mV1(T11A/V7I) plasmids and transfected by control (pMX-U6), CD9, or CD81 short hairpin RNA (shRNA), and the SIRPα, CD81, CD9, and Alix protein expression in extracellular vesicles derived from the transfected stable HEK293 cells; and

FIG. 16 illustrates the comparison result of efficiency of protein EV sorting according to the addition of a few amino acids before and after the mV1(T11A/V7I) while possessing ESM.

### DETAILED DESCRIPTION

Unless defined otherwise, all technical and scientific terms used herein have the meaning commonly understood by a person skilled in the art to which this invention belongs. As used herein, the following terms have the meanings ascribed to them below.

As used herein, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, the term “a combination thereof” or “combinations thereof” refers to all permutations and combinations of the listed items preceding the term. For example, “A, B, C, or a combination thereof” or “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “about” is used to indicate that a value includes the inherent variation of error for the composition, the method used to administer the composition, or the variation that exists among the study subjects.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein, the term “DNA construct” refers to a DNA sequence cloned in accordance with standard cloning procedures used in genetic engineering to relocate a segment of DNA from its natural location to a different site where it will be reproduced. The cloning process involves excision and isolation of the desired DNA segment, insertion of the piece of DNA into the vector molecule, and incorporation of the recombinant vector into a cell where multiple copies or clones of the DNA segment will be replicated. In some embodiments, the DNA construct disclosed herein may comprise a non-naturally occurring DNA molecule which can either be provided as an isolate or integrated in another DNA molecule e.g. in an expression vector or the chromosome of a eukaryotic host cell.

As used herein, the term “vector” refer to carrier DNA molecules or DNA construct for introducing a desired gene into host cells, and amplifying and expressing the desired gene. Preferably, vectors have auxotrophic genes, and have known restriction sites and the ability to replicate in hosts. In general, vectors may comprise a promoter, an enhancer, a terminator, SD sequence, translation initiation and termination codons, and a replication origin. If required, vectors may further comprise selection markers for selecting cells to which the vectors have been introduced. Such selection markers include: genes resistant to drugs such as ampicillin, tetracycline, kanamycin, chloramphenicol, neomycin, hygromycin, puromycin, and zeocin; markers that allow the selection using as an indicator an activity of an enzyme such as galactosidase; and markers such as GFP that allow selection using fluorescence emission as an indicator. It is also possible to use selection markers that allow selection using as an indicator a surface antigen such as EGF receptor and B7-2. By using such selection markers, only cells into which vectors have been introduced, more specifically cells into which the vectors of the present invention have been introduced, can be selected. The vectors may comprise signal sequences for polypeptide secretion. There is no limitation on the type of vectors to be used in the present invention; any vector may be used. In some embodiments, the vector is selected from the group consisting of a pET-vector, a pBAD-vector, a pK184-vector, a pMONO-vector, a pSELECT-vector, pSELECT-Tag-vector, a pVITRO-vector, a pVIVO-vector, a pORF-vector, a pBLAST-vector, a pUNO-vector, a pDUO-vector, a pZERO-vector, a pDeNy-vector, a pDRIVE-vector, a pDRIVE-SEAP-vector, a HaloTag®Fusion-vector, a pTARGET™-vector, a Flexi®-vector, a pDEST-vector, a pHIL-vector, a pPIC-vector, a pMET-vector, a pPink-vector, a pLP-vector, a pTOPO-vector, a pBud-vector, a pCEP-vector, a pCMV-vector, a pDisplay-vector, a pEF-vector, a pFL-vector, a pFRT-vector, a pFastBac-vector, a pGAPZ-vector, a pIZ/V5-vector, a pLenti6-vector, a pMIB-vector, a pOG-vector, a pOpti-vector, a pREP4-vector, a pRSET-vector, a pSCREEN-vector, a pSecTag-vector, a pTEF1-vector, a pTracer-vector, a pTrc-vector, a pUB6-vector, a pVAX1-vector, a pYC2-vector, a pYES2-vector, a pZeo-vector, a pcDNA-vector, a pFLAG-vector, a pTAC-vector, a pT7-vector, a Gateway®-vector, a pQE-vector, a pLEXY-vector, a pRNA-vector, a pPK-vector, a pUMVC-vector, a pLIVE-vector, a pCRUZ-vector, a Duet-vector, and other vectors or derivatives thereof.

As used herein, the term “extracellular vesicle” refers to a cell-derived vesicle comprising a membrane that encloses an internal space. Extracellular vesicles comprise all membrane-bound vesicles that have a smaller diameter than the cell from which they are derived. Generally, extracellular vesicles range in diameter from 20 nm to 1000 nm, and can comprise various macromolecular cargo either within the internal space, displayed on the external surface of the extracellular vesicle, and/or spanning the membrane. The cargo can comprise small molecules, nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein, the term “exosome” refers to a cell-derived nanovesicle comprising a lipid bilayer membrane that encloses an internal space, and which is generated from said cell by direct plasma membrane budding or by fusion of the late endosome with the plasma membrane. The exosome comprises lipid or fatty acid and polypeptide and optionally comprises a therapeutic active payload, a receiver (e.g., a targeting moiety), a polynucleotide (e.g., a nucleic acid, RNA, or DNA), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of an extracellular vesicle.

As used herein, the term “surface-engineered extracellular vesicle” refers to an extracellular vesicle with a membrane modified in its composition. For example, the surface-engineered extracellular vesicle may have a scaffold protein or peptide on the surface of the extracellular vesicle at a higher (or lower) density than a naturally occurring extracellular vesicle does. In accordance with embodiments of the present invention, a surface-engineered extracellular vesicle can be produced from a genetically-engineered producer cell or a progeny thereof. For example, a surface-engineered extracellular vesicle can be produced from a cell transformed or transfected with an exogenous sequence or a DNA construct encoding the scaffold protein or peptide. In some embodiments, the producer cell can be a cell transformed or transfected with both an exogenous sequence or a DNA construct encoding the scaffold protein or peptide and an exogenous sequence or a DNA construct encoding a therapeutic active payload. In some embodiments, the exogenous sequence or DNA construct encoding the scaffold protein or peptide and the exogenous sequence or DNA construct encoding a therapeutic active payload can be introduced into the producer cell by different vectors. In some embodiments, the exogenous sequence or DNA construct encoding the scaffold protein or peptide and the exogenous sequence or DNA construct encoding a therapeutic active payload can be introduced into the producer cell by the same vector. In some embodiments, the scaffold protein or peptide and the therapeutic active payload can be fusion proteins. In some embodiments, the surface-engineered extracellular vesicle can further include a targeting moiety that can be used to target the extracellular vesicle to a desired organ, tissue, or cell. Non-limiting examples of the targeting moiety include an antibody, an antigen-binding fragment of the antibody, an antigen-binding variant of the antibody, an antigen-binding fragment of the antigen-binding variant of the antibody, and an antigen-binding variant of the antigen-binding fragment of the antibody. In some embodiments, the surface-engineered extracellular vesicles in accordance with embodiments of the present invention have better characteristics than surface-engineered extracellular vesicles known in the art. For example, the surface-engineered extracellular vesicles produced by cells introduced with exogenous sequence or DNA construct encoding the scaffold proteins or peptides of the present invention have the scaffold proteins or peptides at a higher density on the surface of the extracellular vesicles than surface-engineered extracellular vesicles known in the art (e.g., extracellular vesicles produced using conventional extracellular vesicle proteins such as PTGFRN).

As used herein, the term “producer cell” or “host cell” refers to a cell used for generating an extracellular vesicle or a surface-engineered extracellular vesicle. A producer cell includes, but is not limited to, a cell known to be effective in generating extracellular vesicles, e.g., HEK293 cells, Chinese hamster ovary (CHO) cells, HeLa cells, and mesenchymal stem cells (MSCs). The producer cell may be transformed or transfected by one or more vectors that contain or contains exogenous sequence(s) or DNA construct(s). In some embodiments of the present invention, the producer cell can be transformed or transfected by one single vector that contains an exogenous sequence or a DNA construct encoding the scaffold protein or peptide of the present invention. In some embodiments, the producer cell can be transformed or transfected by one single vector that contains an exogenous sequence or a DNA construct encoding the scaffold protein or peptide of the present invention and an exogenous sequence or a DNA construct encoding a therapeutically active payload. In some embodiments, the producer cell can be transformed or transfected by a vector that contains an exogenous sequence or a DNA construct encoding the scaffold protein or peptide of the present invention and another vector that contains an exogenous sequence or a DNA construct encoding a therapeutically active payload. In some embodiments, the producer cell can be transformed or transfected with at least one additional exogenous sequence or DNA construct encoding another protein or peptide (e.g., a targeting moiety). The additional exogenous sequence can be introduced into the vector that contains an exogenous sequence or a DNA construct encoding the scaffold protein or peptide of the present invention, an exogenous sequence or a DNA construct encoding a therapeutically active payload, or both. In some embodiments, the exogenous sequence or DNA construct encoding a therapeutically active payload, the additional exogenous sequence or DNA construct encoding another protein or peptide, or both can be introduced into the producer cell so as to modulate endogenous gene expression of the producer cell. In some embodiments, the exogenous sequence or DNA construct encoding a therapeutically active payload, the additional exogenous or DNA construct sequence encoding another protein or peptide, or both can be introduced into the producer cell so as to produce the surface-engineered extracellular vesicle that contains the therapeutically active payload, the another protein or peptide, or both on the surface of the extracellular vesicle.

As used herein, the term “scaffold,” “scaffold protein,” or “scaffold peptide” refers to a protein or peptide that can be targeted to the surface of an extracellular vesicle. In some embodiments, the scaffold proteins or peptides may be located or positioned or comprised in/on the membrane of extracellular vesicle. Scaffold proteins or peptides known in the art include tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin, syndecan, synaptotagmin, apoptosis-linked gene 2-interacting protein X (ALIX), syntenin, PTGFRN, a fragment or variant thereof, a variant of the fragment, and a fragment of the variant. The scaffold proteins or peptides in accordance with embodiments of the present invention comprise the amino acid sequence including G-a-S-b-X1-c-X2, wherein, X1 represents G, A, S or T; X2 represents G or S; a represents 3-4 amino acids; b represents 2-3 amino acids; and c represents 6-7 amino acids, in which G represents glycine, S represents serine, A represents alanine and T represents threonine. In some embodiments of the present invention, the scaffold can be a non-mutant protein or peptide (i.e., a protein or peptide that is naturally targeted to an exosome membrane), a fragment of the non-mutant protein or peptide, a variant of the non-mutant protein or peptide, a fragment of the variant of the non-mutant protein or peptide, or a variant of the fragment of the non-mutant protein or peptide. In some embodiments, the scaffold can be a mutant protein or peptide (i.e., a protein or peptide that is modified to be targeted to an exosome membrane), a fragment of the mutant protein or peptide, a variant of the mutant protein or peptide, a fragment of the variant of the mutant protein or peptide, or a variant of the fragment of the mutant protein or peptide. In some embodiments, the scaffold can be fused to another moiety including, for example, a flag tag, a therapeutic peptide, a targeting moiety, or the like. In some embodiments, the scaffold can comprise a transmembrane protein, a peripheral protein, or a soluble protein. In some embodiments, the scaffold can be attached to the membrane of an extracellular vesicle by a linker.

Scaffolds, fragments of the scaffolds, variants of the scaffolds, fragments of the variants of the scaffolds, and variants of the fragments of the scaffolds in accordance with embodiments of the present invention have the ability to be specifically targeted to the surface of extracellular vesicles. In some embodiments, the Scaffolds, fragments of the scaffolds, variants of the scaffolds, fragments of the variants of the scaffolds, and variants of the fragments of the scaffolds in accordance with embodiments of the present invention may be located or positioned or comprised in/on the membrane of extracellular vesicle. As used herein, the term “a fragment” of a protein, peptide, or nucleic acid refers to a segment of the protein, peptide, or nucleic acid. As used herein, the term “variant” of a protein, peptide, or nucleic acid refers to a protein, peptide, or nucleic acid having has at least one amino acid or nucleotide which is different from the protein, peptide, or nucleic acid. A variant of a protein, peptide, or nucleic acid includes, but is not limited to, a substitution, deletion, frameshift, or rearrangement in the protein, peptide, or nucleic acid. The term may be used interchangeably with the term “mutant.”

The fragments of the scaffolds in accordance with some embodiments of the present invention may retain at least 30%, 35%, 40%, 45%, 50%, 55%, 60%, 65%, 70%, 75%, 80%, 85%, 90%, 95%, 99%, or 100% of the ability of the scaffolds to be specifically targeted to extracellular vesicles. The variants of the scaffolds in accordance with some embodiments of the present invention may retain at least 30%, 35%, 40%, 45%, 50%, 55%, 60%, 65%, 70%, 75%, 80%, 85%, 90%, 95%, 99%, or 100% of the ability of the scaffolds to be specifically targeted to extracellular vesicles. The fragments of the variants of the scaffolds may retain at least 30%, 35%, 40%, 45%, 50%, 55%, 60%, 65%, 70%, 75%, 80%, 85%, 90%, 95%, 99%, or 100% of the ability of the variants of the scaffolds to be specifically targeted to extracellular vesicles. The variants of the fragments of the scaffolds may retain at least 30%, 35%, 40%, 45%, 50%, 55%, 60%, 65%, 70%, 75%, 80%, 85%, 90%, 95%, 99%, or 100% of the ability of the fragments of the scaffolds to be specifically targeted to extracellular vesicles.

The variants of the scaffolds in accordance with some embodiments of the present invention may have at least 50%, 55%, 60%, 65%, 70%, 75%, 80%, 85%, 90%, 95%, 99%, or 100% identity to the scaffolds. The variants of the fragments of the scaffolds in accordance with some embodiments of the present invention may have at least 50%, 55%, 60%, 65%, 70%, 75%, 80%, 85%, 90%, 95%, 99%, or 100% identity to the fragments of the scaffolds.

As used herein, the term “target protein” and “target peptide” can be used interchangeably and refers to a protein or a peptide of interest to be delivered, expressed or introduced at the surface, on the surface or inside the membrane of extracellular vesicles. In some embodiments, the target protein or target peptide may be delivered, expressed or introduced at the surface, on the surface or inside the membrane of extracellular vesicles by fused to the scaffold protein or scaffold peptide. In some embodiments, the target protein or target peptide may be fused to the N-terminal or C-terminal of the scaffold protein or scaffold peptide. In some embodiments, the target protein or target peptide may be fused to the scaffold protein or scaffold peptide via linker peptide. In some embodiments, the target proteins or target peptides may be therapeutic proteins, antigens, cytokines, ligands, receptors, immunoglobulins, a marker polypeptide (e.g., a label protein, such as Green Fluorescent Protein, or an enzyme, for instance), enzymes, ionic channels, etc., or a portion thereof. In some embodiments, the target protein or target peptide may be a therapeutic molecule or biologically active molecule.

As used herein, the term “biologically active molecule” and “therapeutic molecule” can be interchangeably used and refers to an agent that has activity in a biological system (e.g., a cell or a human subject), including, but not limited to a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof can be natural, synthetic or humanized, a peptide hormone, a receptor, a signaling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which can be modified or unmodified; an amino acid or analogue thereof, which can be modified or unmodified; a non-peptide (e.g., steroid) hormone; a proteoglycan; a lipid; or a carbohydrate. In certain aspects, a biologically active molecule comprises a therapeutic molecule (e.g., an antigen), a targeting moiety (e.g., an antibody or an antigen-binding fragment thereof), an adjuvant, an immune modulator, or any combination thereof. In some embodiments, the biologically active molecule comprises a macromolecule (e.g., a protein, an antibody, an enzyme, a peptide, DNA, RNA, or any combination thereof). In some embodiments, the biologically active molecule comprises a small molecule (e.g., an antisense oligomer (ASO), a phosphorodiamidate morpholino oligomer (PMO), a peptide-conjugated phosphorodiamidate morpholino oligomer (PPMO), an siRNA, STING, a pharmaceutical drug, or any combination thereof). In some embodiments, the biologically active molecules are exogenous to the extracellular vesicles, i.e., not naturally found in the extracellular vesicles. In some embodiments, the biologically active molecule or therapeutic molecule may be a therapeutic protein or therapeutic peptide.

As used herein, the term “linker” refers to any molecular structure that can conjugate a peptide or a protein to another molecule (e.g., a different peptide or protein, a small molecule, etc.). Suitable linkers are well known to those of skill in the art and include, but are not limited to, straight or branched-chain carbon linkers, heterocyclic carbon linkers, or peptide linkers (see, e.g., Chen et al., Advanced Drug Delivery Reviews, 2013, Vol. 65:10, pp. 1357-1369). The linkers can be joined to the carboxyl and amino terminal amino acids through their terminal carboxyl or amino groups or through their reactive side-chain groups. In addition, in some embodiments, linkers can be classified as flexible or rigid, and they can be cleavable (e.g., comprise one or more protease-cleavable sites, which can be located within the sequence of the linker or flanking the linker at either end of the linker sequence).

As used herein, the term “payload” refers to an agent capable of acting on a target (e.g., a target cancer cell) that is contacted with an extracellular vesicle. In some embodiments, the payload can be introduced into an extracellular vesicle. In some embodiments, the payload can be introduced into a producer cell. Non-limiting examples of the payload include nucleotides, nucleic acids (e.g., DNA mRNA, miRNA, dsDNA, lncRNA, and siRNA), amino acids, polypeptides, lipids, carbohydrates, and small molecules. In preferred embodiment, the payload may be a therapeutically or biologically active agent.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired extracellular vesicles, that have undergone one or more processes of purification, e.g., a selection or an enrichment of the desired extracellular vesicle preparation. In some embodiments, isolating or purifying as used herein is the process of removing, partially removing (e.g., a fraction) of the extracellular vesicles from a sample containing producer cells. In some embodiments, an isolated extracellular vesicle composition has no detectable undesired activity or, alternatively, the level or amount of the undesired activity is at or below an acceptable level or amount. In other embodiments, an isolated extracellular vesicle composition has an amount and/or concentration of desired extracellular vesicles at or above an acceptable amount and/or concentration. In other embodiments, the isolated extracellular vesicle composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some embodiments, isolated extracellular vesicle preparations are substantially free of residual biological products. In some embodiments, the isolated extracellular vesicle preparations are 100% free, 99% free, 98% free, 97% free, 96% free, or 95% free of any contaminating biological matter. Residual biological products can include abiotic materials (including chemicals) or unwanted nucleic acids, proteins, lipids, or metabolites. Substantially free of residual biological products can also mean that the extracellular vesicle composition contains no detectable producer cells and that only extracellular vesicles are detectable.

As used herein, the term “pharmaceutically acceptable” refers to compounds and compositions which are suitable for administration to humans and/or animals without undue adverse side effects such as toxicity, irritation and/or allergic response commensurate with a reasonable benefit/risk ratio.

As used herein, the term “biologically active” refers to the ability to modify the physiological system of an organism without reference to how the active agent has its physiological effects.

As used herein, the terms “subject” and “patient” are used interchangeably herein and will be understood to encompass mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fishes, and the like.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “administration” or “administering” of a composition refers to providing a composition to a subject in need of treatment. In accordance with embodiments of the present invention, therapeutic compositions may be administered singly or in combination with one or more additional therapeutic agents. The methods of administration of such compositions may include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration or optic administration.

As used herein, the terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art. The compositions of the present disclosure may be administered by way of known pharmaceutical formulations, including tablets, pills, capsules, a liquid, an inhalant, a nasal spray solution, a suppository, a solution, a gel, an emulsion, an ointment, eye drops, ear drops, and the like.

As used herein, the term “effective amount” or “therapeutically effective amount” refer to a sufficient amount of an active ingredient(s) described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a surface-engineered exosome as disclosed herein required to provide a clinically significant decrease in disease symptoms. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skills in the art using routine experimentation based on the information provided herein.

In an aspect, the present invention provides a DNA construct comprising a DNA sequence encoding a scaffold peptide, wherein the amino acid sequence of the scaffold peptide includes a sequence represented by G-a-S-b-X1-c-X2, in which X1 represents G, A, S or T; X2 represents G or S; a represents 3-4 amino acids; b represents 2-3 amino acids; c represents 6-7 amino acids; G represents glycine; S represents serine; A represents alanine; and T represents threonine.

In some embodiments, in the sequence of G-a-S-b-X1-c-X2, X1 and X2 may be G and G, G and S, A and G, A and S, S and G, S and S, T and G, or T and S, respectively.

In some embodiments, the sequence G-a-S-b-X1-c-X2 may have 16 amino acids. For example, a may represent 3 amino acids, b may represent 3 amino acids and c may represent 6 amino acids. Also, for example, a may represent 4 amino acids, b may represent 2 amino acids and c may represent 6 amino acids. In addition, for example, a may represent 3 amino acids, b may represent 2 amino acids and c may represent 7 amino acids.

In some embodiments, the amino acids a, b, and c may include V, G, L, I, A, T, S, C, F, W, Y, and P, in which V represents valine, G represents glycine, L represents leucine, I represents isoleucine, A represents alanine, T represents threonine, S represents serine, C represents cysteine, F represents phenylalanine, W represents tryptophan, Y represents tyrosine, and P represents proline.

In some embodiments, a may represent 3-4 amino acids selected from the group consisting of V, G, L, I and A, in which V represents valine, G represents glycine, L represents leucine, I represents isoleucine, and A represents alanine. Non-limiting examples of the 3-4 amino acids include: VGL, VGI, VGT, VGA, VLG, VLI, VLT, VLA, VIG, VIL, VIT, VIA, VTG, VTL, VTI, VTA, VAG, VAL, VAI, VAT, GVL, GVI, GVT, GVA, GLV, GLI, GLT, GLA, GIV, GIL, GIT, GIA, GTV, GTL, GTI, GTA, GAV, GAL, GAI, GAT, LVG, LVI, LVT, LVA, LGV, LGI, LGT, LGA, LIV, LIG, LIT, LIA, LTV, LTG, LTI, LTA, LAV, LAG, LAI, LAT, IVG, IVL, IVT, IVA, IGV, IGL, IGT, IGA, ILV, ILG, ILT, ILA, ITV, ITG, ITL, ITA, IAV, IAG, IAL, IAT, TVG, TVL, TVI, TVA, TGV, TGL, TGI, TGA, ILV, TLG, TLI, TLA, TIV, TIG, TIL, TIA, TAV, TAG, TAL, TAI, AVG, AVL, AVI, AVT, AGV, AGL, AGI, AGT, ALV, ALG, ALI, ALT, AIV, AIG, AIL, AIT, ATV, ATG, ATL, ATI, VGLI, VGLT, VGLA, VGIL, VGIT, VGIA, VGTL, VGTI, VGTA, VGAL, VGAI, VGAT, IGVL, IGVT, IGVA, IGLV, IGLT, IGLA, IGTV, IGTL, IGTA, IGAV, IGAL, or IGAT.

In some embodiments, b may represent 2-3 amino acids selected from the group consisting of V, I, A, and T, in which V represents valine, I represents isoleucine, A represents alanine and T represents threonine. Non-limiting examples of the 2-3 amino acid sequences include: VI, VA, VT, IV, IA, IT, AV, AI, AT, TV, TI, TA, VV, II, AA, TT, VIA, VIT, VAI, VAT, IVA, IVT, IAV, ITV, ITA, AVI, AVT, AIV, AIT, ATV, ATI, TVI, TVA, ITV, TIA, TAV, and TAI.

In some embodiments, c may represent 6-7 amino acids selected from the group consisting of L, S, C, and I, in which L represents leucine, S represents serine, C represents cysteine and I represents isoleucine. Non-limiting examples of the 6-7 amino acids include: LLSCLI, LLSCIL, LLSLCI, LLSLIC, LLCSLI, LLCSIL, LLCISL, LLCILS, LLSICL, LLSILC, LSLCLI, LSLCIL, LSLLCI, LSLLIC, LSLCLI, LSLCIL, ILLSCLI, ILLSCIL, ILLSLCI, ILLSLIC, ILLCSLI, ILLCSIL, ILLCISL, ILLCILS, ILLSICL, ILLSILC, ILSLCLI, ILSLCIL, ILSLLCI, ILSLLIC, ILSLCLI, ILSLCIL, LILSCLI, LILSCIL, LILSLCI, LILSLIC, LILCSLI, LILCSIL, LILCISL, LILCILS, LILSICL, LILSILC, LISLCLI, LISLCIL, LISLLCI, LISLLIC, LISLCLI, and LISLCII.

In some embodiments, the sequence G-a-S-b-X1-c-X2 may be one of the amino acid sequences represented by ESM SEQ ID NOS: 1-14.

(ESM SEQ ID NO: 1)
GVGLSTVIGLLSCLIG
(ESM SEQ ID NO: 2)
GIGLSTVIGLLSCLIG
(ESM SEQ ID NO: 3)
GVGLSAVIGLLSCLIG
(ESM SEQ ID NO: 4)
GIGLSAVIGLLSCLIG
(ESM SEQ ID NO: 5)
GILLSAVIGLLSCLIG
(ESM SEQ ID NO: 6)
GIGLSLVIGLLSCLIG
(ESM SEQ ID NO: 7)
GIGLSAVIGLLLCLIG
(ESM SEQ ID NO: 8)
GIGLSAVIGLLSLLIG
(ESM SEQ ID NO: 9)
GIGLSAVIALLSCLIG
(ESM SEQ ID NO: 10)
GIGLSAVISLLSCLIG
(ESM SEQ ID NO: 11)
GIGLSAVITLLSCLIG
(ESM SEQ ID NO: 12)
GIGLSAVIGLLSCLIS
(ESM SEQ ID NO: 13)
GIGLASVIGLLSCLIG
(ESM SEQ ID NO: 14)
GIGLSAVGILLSCLIG

Non-limiting examples of the sequence G-a-S-b-X1-c-X2 include:

| Amino acid sequence | ESM SEQ ID NO |
|---|---|
| GVGLSTVIALLSCLIG | 15 |
| GVGLSTVISLLSCLIG | 16 |
| GVGLSTVITLLSCLIG | 17 |
| GVGLSTVIGLLSCLIS | 18 |
| GVGLSTVIALLSCLIS | 19 |
| GVGLSTVISLLSCLIS | 20 |
| GVGLSTVITLLSCLIS | 21 |
| GIGLSTVIALLSCLIG | 22 |
| GIGLSTVISLLSCLIG | 23 |
| GIGLSTVITLLSCLIG | 24 |
| GIGLSTVIALLSCLIS | 25 |
| GIGLSTVISLLSCLIS | 26 |
| GIGLSTVITLLSCLIS | 27 |
| GVGLSAVIALLSCLIG | 28 |
| GVGLSAVISLLSCLIG | 29 |
| GVGLSAVITLLSCLIG | 30 |
| GVGLSAVIGLLSCLIS | 31 |
| GVGLSAVIALLSCLIS | 32 |
| GVGLSAVISLLSCLIS | 33 |
| GVGLSAVITLLSCLIS | 34 |
| GILLSAVIALLSCLIG | 35 |
| GILLSAVISLLSCLIG | 36 |
| GILLSAVITLLSCLIG | 37 |
| GILLSAVIGLLSCLIS | 38 |
| GILLSAVIALLSCLIS | 39 |
| GILLSAVISLLSCLIS | 40 |
| GILLSAVITLLSCLIS | 41 |
| GIGLSLVIALLSCLIG | 42 |
| GIGLSLVISLLSCLIG | 43 |
| GIGLSLVITLLSCLIG | 44 |
| GIGLSLVIGLLSCLIS | 45 |
| GIGLSLVIALLSCLIS | 46 |
| GIGLSLVISLLSCLIS | 47 |
| GIGLSLVITLLSCLIS | 48 |
| GIGLSAVIALLLCLIG | 49 |
| GIGLSAVISLLLCLIG | 50 |
| GIGLSAVITLLLCLIG | 51 |
| GIGLSAVIGLLLCLIS | 52 |
| GIGLSAVIALLLCLIS | 53 |
| GIGLSAVISLLLCLIS | 54 |
| GIGLSAVITLLLCLIS | 55 |
| GIGLSAVIALLSLLIG | 56 |
| GIGLSAVISLLSLLIG | 57 |
| GIGLSAVITLLSLLIG | 58 |
| GIGLSAVIGLLSLLIS | 59 |
| GIGLSAVIALLSLLIS | 60 |
| GIGLSAVISLLSLLIS | 61 |
| GIGLSAVITLLSLLIS | 62 |
| GIGLASVIALLSCLIG | 63 |
| GIGLASVISLLSCLIG | 64 |
| GIGLASVITLLSCLIG | 65 |
| GIGLASVIGLLSCLIS | 66 |
| GIGLASVIALLSCLIS | 67 |
| GIGLASVISLLSCLIS | 68 |
| GIGLASVITLLSCLIS | 69 |
| GIGLSAVAILLSCLIG | 70 |
| GIGLSAVSILLSCLIG | 71 |
| GIGLSAVTILLSCLIG | 72 |
| GIGLSAVGILLSCLIS | 73 |
| GIGLSAVAILLSCLIS | 74 |
| GIGLSAVSILLSCLIS | 75 |
| GIGLSAVTILLSCLIS | 76 |
| GIGLASAVIGLLSCLIG | 77 |
| GIGLASAVIALLSCLIG | 78 |
| GIGLASAVISLLSCLIG | 79 |
| GIGLASAVITLLSCLIG | 80 |
| GIGLASAVIGLLSCLIS | 81 |
| GIGLASAVIALLSCLIS | 82 |
| GIGLASAVISLLSCLIS | 83 |
| GIGLASAVITLLSCLIS | 84 |
| GIGLSAVIGILLSCLIG | 85 |
| GIGLSAVIAILLSCLIG | 86 |
| GIGLSAVISILLSCLIG | 87 |
| GIGLSAVITILLSCLIG | 88 |
| GIGLSAVIGILLSCLIS | 89 |
| GIGLSAVIAILLSCLIS | 90 |
| GIGLSAVISILLSCLIS | 91 |
| GIGLSAVITILLSCLIS | 92 |
| GIGLASAVIGILLSCLIG | 93 |
| GIGLASAVIAILLSCLIG | 94 |
| GIGLASAVISILLSCLIG | 95 |
| GIGLASAVITILLSCLIG | 96 |
| GIGLASAVIGILLSCLIS | 97 |
| GIGLASAVIAILLSCLIS | 98 |
| GIGLASAVISILLSCLIG | 99 |
| GIGLASAVITILLSCLIG | 100 |

In some embodiments, the scaffold peptide may further comprise KYPLLI at the N-terminal of the sequence G-a-S-b-X1-c-X2, in which K represents lysine, Y represents tyrosine, P represents proline, L represents leucine and I represents isoleucine.

In some embodiments, the scaffold peptide may further comprise FKYPLLI, AFKYPLLI, NAFKYPLLI, LNAFKYPLLI, VLNAFKYPLLI or DVLNAFKYPLLI at the N-terminal of the sequence G-a-S-b-X1-c-X2, in which D represents aspartic acid, V represents valine, L represents leucine, N represents asparagine, A represents alanine, F represents phenylalanine, K represents lysine, Y represents tyrosine, P represents proline, L represents leucine, and I represents isoleucine.

In some embodiments, the scaffold peptide may further comprise YCSS at the C-terminal of the sequence G-a-S-b-X1-c-X2, in which Y represents tyrosine, C represents cysteine, and S represents serine.

In some embodiments, the scaffold peptide may further comprise YCSSH, YCSSHW, YCSSHWC, YCSSHWCC, YCSSHWCCK, YCSSHWCCKK, YCSSHWCCKKE, YCSSHWCCKKEV, YCSSHWCCKKEVQ, YCSSHWCCKKEVQE, YCSSHWCCKKEVQET, YCSSHWCCKKEVQETR, YCSSHWCCKKEVQETRR, YCSSHWCCKKEVQETRRE, YCSSHWCCKKEVQETRRER, YCSSHWCCKKEVQETRRERR, YCSSHWCCKKEVQETRRERRR, YCSSHWCCKKEVQETRRERRRL, YCSSHWCCKKEVQETRRERRRLM, YCSSHWCCKKEVQETRRERRRLMS, YCSSHWCCKKEVQETRRERRRLMSM, YCSSHWCCKKEVQETRRERRRLMSME, YCSSHWCCKKEVQETRRERRRLMSMEM or YCSSHWCCKKEVQETRRERRRLMSMEMD at the C-terminal of the sequence G-a-S-b-X1-c-X2, in which Y represents tyrosine, C represents cysteine, S represents serine, H represents histidine, W represents tryptophan, K represents lysine, E represents glutamic acid, V represents valine, Q represents glutamine, T represents threonine, R represents arginine, L represents leucine, M represents methionine, and D represents aspartic acid.

In some embodiments, the scaffold peptide may further comprise YCSSHWC at the C-terminal of the sequence G-a-S-b-X1-c-X2, in which Y represents tyrosine, C represents cysteine, S represents serine, H represents histidine, and W represents tryptophan.

In some embodiments, the scaffold peptide may be one of the amino acid sequences as set forth in SEQ ID NOS: 101-142.

In some embodiments, the DNA construct may further comprise a DNA sequence encoding an amino acid sequence of a target protein. In some embodiments, the target protein may be a therapeutic protein. In some embodiments, the target protein may be fused to the scaffold peptide.

In another aspect, the present invention provides a vector comprising the DNA construct described above. The vector may be a plasmid, a phage, a virus, an artificial chromosome, etc. Typical examples include plasmids, such as those derived from commercially available plasmids, in particular pUC, pcDNA, pBR, etc. Other examples are vectors derived from viruses, such as replication defective retroviruses, adenoviruses, AAV, baculoviruses or vaccinia viruses. The choice of the vector may be adjusted by the skilled person depending on the recombinant host cell in which said vector should be used. Without intending to limit the scope of the invention, for example, vectors that can transfect or infect mammalian cells can be chosen.

In still another aspect, the present invention provides a host cell comprising the vector described above. In some embodiments, the host cell may produce an extracellular vesicle comprising the scaffold peptide described above on the surface thereof. The cells may be cultured and maintained in any appropriate medium, such as RPMI, DMEM, etc. The cultures may be performed in any suitable device, such as plates, dishes, tubes, flasks, etc. The vector can be introduced into the host cell by any conventional method, such as by naked DNA technique, cationic lipid-mediated transfection, polymer-mediated transfection, peptide-mediated transfection, virus-mediated infection, physical or chemical agents or treatments, electroporation, etc. In this regard, it should be noted that transient transfection is sufficient to express the gene (i.e, DNA construct of the present invention) so that it is not necessary to create stable cell lines or to optimize the transfection conditions.

In yet another aspect, the present invention provides an extracellular vesicle comprising the scaffold peptide encoded by the DNA construct of the present invention described above. In some embodiments, the extracellular vesicle may be a surface-engineered. In some embodiments, the surface-engineered and/or lumen engineered extracellular vesicles may be generated by chemical and/or physical methods, such as PEG-induced fusion and/or ultrasonic fusion. In other embodiments, the surface-engineered extracellular vesicles are generated by genetic engineering. Extracellular vesicles produced from a genetically-modified producer cell or a progeny of the genetically-modified cell can contain modified membrane compositions. In some embodiments, the genetically-modified producer cell or progeny of the genetically-modified cell may comprise one or more exogenous proteins(peptides) that are not naturally found in the cell. In certain aspects, the one or more exogenous proteins may be scaffold proteins or peptides, such as the scaffold peptide disclosed herein. In some embodiments, surface-engineered extracellular vesicles may have the scaffold peptide disclosed herein at a higher density compared to the density of other scaffold proteins or peptides such as tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin, syndecan, synaptotagmin, apoptosis-linked gene 2-interacting protein X (ALIX), syntenin, PTGFRN, a fragment or variant thereof, a variant of the fragment, and a fragment of the variant. For example, surface-engineered extracellular vesicles can be produced from host cells or producer cells transformed with an exogenous sequence encoding the DNA construct disclosed herein. Extracellular vesicles comprising peptides or proteins expressed from the exogenous sequence (e.g., DNA construct described herein) can include modified membrane protein compositions.

In some embodiments, the scaffold peptide described herein that are capable of anchoring a cargo or target protein (or peptide) such as exogenously biologically active molecules (e.g., those disclosed herein) can be used in constructing a surface-engineered extracellular vesicles.

Fusion proteins can be also comprised on the surface of the extracellular vesicles; for example, the scaffold peptide described herein fused to an affinity tag (e.g., His tag, GST tag, glutathione-S-transferase, S-peptide, HA, Myc, FLAG™ (Sigma-Aldrich Co.), MBP, SUMO, and Protein A) can be used for purification or removal of the surface-engineered extracellular vesicles with a binding agent specific to the affinity tag.

Fusion proteins having a therapeutic activity can be also used for generating surface-engineered extracellular vesicles. Accordingly, in some embodiments, extracellular vesicles disclosed herein can be engineered or modified to express the fusion protein and can be used to deliver one or more (e.g., two, three, four, five or more) therapeutic molecules to a target. For example, the fusion protein may comprise the scaffold peptide described herein and a therapeutic substance (e.g., peptide or protein). In some embodiments, the therapeutic substance may be fused directly to the scaffold peptide described herein. In some embodiments, the therapeutic substance may be anchored to the scaffold peptide described herein via a linker.

In some embodiments, the linker may be a peptide linker. In some embodiments, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids. In some embodiments, the peptide linker may be synthetic, i.e., non-naturally occurring. In some embodiments, a peptide linker may include peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in some embodiments the peptide linker can comprise non-naturally occurring polypeptides which are modified forms of naturally occurring polypeptides (e.g., comprising a mutation such as an addition, substitution, or deletion). Linkers can be susceptible to cleavage (“cleavable linker”) thereby facilitating release of the exogenous biologically active molecule. In some embodiments, the linker may comprise a non-cleavable linker.

In some embodiments, the biologically active molecule (e.g., therapeutic peptide or protein) may be selected from the group consisting of a natural peptide, a recombinant peptide, a synthetic peptide, and a linker to a therapeutic substance. The therapeutic substance can be nucleotides, amino acids, lipids, carbohydrates, or small molecules. The therapeutic peptide can be an antibody, an enzyme, a ligand, a receptor, an antimicrobial peptide, or a fragment or variant thereof. In some embodiments, the therapeutic peptide may be a nucleic acid binding protein. The nucleic acid binding protein can be Dicer, an Argonaute protein, TRBP, or MS2 bacteriophage coat protein. In some embodiments, the nucleic acid binding protein may additionally comprise one or more RNA or DNA molecules. The one or more RNA can be a miRNA, siRNA, antisense oligonucleotide, phosphorodiamidate morpholino oligomer (PMO), peptide-conjugated phosphorodiamidate morpholino oligomer (PPMO), guide RNA, lincRNA, mRNA, antisense RNA, dsRNA, or any combination thereof. In some embodiments, the biologically active molecule may be a part of a protein-protein interaction system. In some embodiments, the biologically active molecule which can be anchored to the scaffold peptide described herein and expressed on a surface of extracellular vesicle may comprise an antigen. In certain embodiments, the antigen may comprise a tumor antigen. Non-limiting examples of tumor antigens include: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen (CTA), MART-1 gp100, TNF-related apoptosis-inducing ligand, Brachyury (preferentially expressed antigen in melanoma (PRAME)), and any combination thereof. In some embodiments, the antigen may be derived from a bacterium, a virus, fungus, protozoa, or any combination thereof. In some embodiments, the antigen may be derived from an oncogenic virus. In further embodiments, the antigen may be derived from the group comprising: a Human Gamma herpes virus 4 (Epstein Barr virus), influenza A virus, influenza B virus, cytomegalovirus, Staphylococcus aureus, Mycobacterium tuberculosis, Chlamydia trachomatis, HIV-1, HIV-2, corona viruses (e.g., MERS-CoV and SARS CoV), filoviruses (e.g., Marburg and Ebola), Streptococcus pyogenes, Streptococcus pneumoniae, Plasmodia species (e.g., vivax and falciparum), Chikungunya virus, Human Papilloma virus (HPV), Hepatitis B, Hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., and any combination thereof.

Non-limiting examples of other suitable biologically active molecules include pharmacologically active drugs and genetically active molecules, including antineoplastic agents, anti-inflammatory agents, hormones or hormone antagonists, ion channel modifiers, and neuroactive agents. Examples of suitable payloads of therapeutic agents include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Suitable payloads further include toxins, and biological and chemical warfare agents, for example see Somani, S. M. (ed.), Chemical Warfare Agents, Academic Press, New York (1992)).

In some embodiments, fusion proteins having a targeting moiety may be used. For example, fusion proteins can comprise the scaffold peptide described herein and a targeting moiety. The targeting moiety can be used for targeting the extracellular vesicle to a specific organ, tissue, or cell for a treatment using the extracellular vesicle. In certain embodiments, the targeting moiety may bind to a marker (or target molecules) expressed on a cell or a population of cells. In certain embodiments, the marker may be expressed on multiple cell types, e.g., all antigen-present cells (e.g., dendritic cells, macrophages, and B lymphocytes). In some embodiments, the marker may be expressed only on a specific population of cells (e.g., dendritic cells). Non-limiting examples of markers that are expressed on specific population of cells (e.g., dendritic cells) include a C-type lectin domain family 9 member A (CLEC9A) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), and any combination thereof. In some embodiments, the targeting moiety may be an antibody or antigen-binding fragment thereof. Antibodies and antigen-binding fragments thereof include whole antibodies, polyclonal, monoclonal and recombinant antibodies, fragments thereof, and they may further include single-chain antibodies, humanized antibodies, murine antibodies, chimeric, mouse-human, mouse-primate, primate-human monoclonal antibodies, anti-idiotype antibodies, antibody fragments (e.g., scFv, (scFv)2, Fab, Fab′, and F(ab′)2, F(ab1)2, Fv, dAb, and Fd fragments), diabodies, and antibody-related polypeptides. Antibodies and antigen-binding fragments thereof may include bispecific antibodies and multispecific antibodies so long as they exhibit the desired biological activity or function.

In some embodiments, the extracellular vesicle may encapsulate a target protein (e.g., therapeutic protein or therapeutic substance such as a nucleotide, an amino acid, a lipid, a carbohydrate, a small molecule, and any combination thereof).

In some embodiments, the extracellular vesicles described herein demonstrate superior characteristics compared to extracellular vesicles known in the art. For example, extracellular vesicles produced by using the scaffold peptide described herein contain modified proteins that are more highly enriched on their surface than extracellular vesicles in the prior art, e.g., those produced using conventional exosome proteins. In some embodiments, the expression level of the modified proteins is increased (i.e., enriched) by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% or more, compared to the expression of the corresponding protein using conventional exosome proteins. Moreover, in some embodiments, the biological activity of the extracellular vesicles of the present disclosure is greater than that of extracellular vesicles known in the art. For example, a surface engineered extracellular vesicle comprising a therapeutic or biologically relevant exogenous sequence fused to the scaffold peptide described herein can have more of the desired engineered characteristics than fusion to scaffolds known in the art. Examples of scaffold proteins known in the art include, but not limited to tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin, syndecan, synaptotagmin, apoptosis-linked gene 2-interacting protein X (ALIX), syntenin, PTGFRN, a fragment or variant thereof, a variant of the fragment, and a fragment of the variant, and peptides that have affinity to any of these proteins or fragments thereof.

In some embodiments, the surface-engineered extracellular vesicle comprising a fusion protein containing an exogenous sequence (e.g., encoding an exogenous biologically active molecule, e.g., antigen, adjuvant, targeting moiety, and/or immune modulator) and the scaffold peptide described herein has a higher density of the fusion protein than similarly engineered extracellular vesicles comprising an exogenous sequence conjugated to a conventional extracellular vesicle protein known in the art (e.g., CD9, CD63, CD81, PDGFR, GPI anchor proteins, lactadherin LAMP2, LAMP2B, syndecan, synaptotagmin, apoptosis-linked gene 2-interacting protein X (ALIX), syntenin, PTGFRN, a fragment or variant thereof, a variant of the fragment, and a fragment of the variant, or a peptide that binds thereto). In some embodiments, the fusion protein containing the scaffold peptide described herein is present at about 2-, about 4-, about 8-, about 16-, about 32-, about 64-, about 100-, about 200-, about 400-, about 800-, about 1,000-fold or a higher density on the extracellular vesicle surface than fusion proteins on other extracellular vesicle surfaces similarly modified using a conventional extracellular vesicle protein.

In some embodiments, the extracellular vesicle described herein can be isolated from a host cell or producer cell comprising the vector described herein. When extracellular vesicles are produced from in vitro cell culture, various producer cells, e.g., HEK293 cells, Chinese hamster ovary (CHO) cells, mesenchymal stem cells (MSCs), HT-1080 cells, MB-231 cells, Raji cells, PER.C6 cells, and CAP cells can be used for the present disclosure. A non-limiting example of the host or producer cell is HEK293 cells.

The producer cell (or host cell) can be genetically modified to comprise one or more exogenous sequences to produce surface-engineered extracellular vesicles. In some embodiments, the one or more exogenous sequences may encode a scaffold peptide described herein. In some embodiments, the one or more exogenous sequences may encode an exogenous biologically active molecule described herein. In some embodiments, the one or more exogenous sequences may encode both the scaffold peptide describe herein and an exogenous biologically active molecule described herein. The genetically-modified producer cell can contain the exogenous sequence introduced by transient or stable transformation. The exogenous sequence can be introduced to the producer cell as a plasmid. The exogenous sequences can be stably integrated into a genomic sequence of the producer cell, at a targeted site or in a random site. In some embodiments, a stable cell line may be generated for production of surface-engineered extracellular vesicles. An exogenous sequence encoding the scaffold peptide described herein can be introduced to produce a surface-engineered extracellular vesicle containing the scaffold peptide. An exogenous sequence encoding an affinity tag can be introduced to produce a surface-engineered extracellular vesicle containing a fusion protein comprising the affinity tag attached to the scaffold peptide. As described herein, in some embodiments, an exogenous sequence encoding an exogenous biologically active molecule can be introduced to produce a surface-engineered extracellular vesicle containing a fusion protein comprising the exogenous biologically active molecule attached (e.g., directly or via a linker) to the scaffold peptide.

In some embodiments, the producer cell (or host cell) may further be modified to comprise an additional exogenous sequence. For example, an additional exogenous sequence can be introduced to modulate endogenous gene expression, or produce an extracellular vesicle including a certain polypeptide as a payload. In some embodiments, the producer cell may be modified to comprise two exogenous sequences, one encoding the scaffold peptide, and the other encoding a payload. In some embodiments, the producer cell can be further modified to comprise an additional exogenous sequence conferring additional functionalities to extracellular vesicles, for example, specific targeting capabilities, delivery functions, enzymatic functions, increased or decreased half-life in vivo, etc. In some embodiments, the producer cell may be modified to comprise two exogenous sequences, one encoding the scaffold peptide, and the other encoding a protein conferring the additional functionalities to extracellular vesicles.

In some embodiments, the producer cell (or host cell) may be modified to comprise two exogenous sequences, each of the two exogenous sequences encoding a fusion protein on the extracellular vesicle surface. In some embodiments, a surface-engineered extracellular vesicle from the producer cell has a higher density of the scaffold peptide compared to native extracellular vesicles isolated from an unmodified cell of the same or similar cell type. In some embodiments, surface-engineered extracellular vesicle contains the scaffold peptide at a density about 2-, about 4-, about 8-, about 16-, about 32-, about 64-, about 100-, about 200-, about 400-, about 800-, about 1,000-fold or higher than a native extracellular vesicle isolated from an unmodified cell of the same or similar cell type.

More specifically, surface-engineered extracellular vesicles can be produced from a cell transformed (or transfected) with a sequence encoding one or more scaffold. Any of the one or more scaffold peptides described herein can be expressed in the producer cell from a plasmid, an exogenous sequence inserted into the genome or other exogenous nucleic acid such as a synthetic messenger RNA (mRNA).

In some embodiments, the scaffold peptide described herein may be fused to one or more heterologous proteins (e.g., exogenous biologically active molecules). In some embodiments, the one or more heterologous proteins may be fused to the N-terminus of the scaffold peptide. In some embodiments, the one or more heterologous proteins may be fused to the C-terminus of the scaffold peptide. In some embodiments, the one or more heterologous proteins may be fused to the N-terminus and the C-terminus of the scaffold peptide.

In still yet another aspect, the present invention provides a pharmaceutical composition comprising the extracellular vesicle described herein, and a pharmaceutically acceptable carrier and/or excipient. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions can be generally formulated sterile and in full compliance with all Good Manufacturing Practice (GMP) regulations of the U.S. Food and Drug Administration. In some embodiments, a pharmaceutical composition may comprise one or more therapeutic agents and an extracellular vesicle described herein. In certain embodiments, the extracellular vesicles may be co-administered with of one or more additional therapeutic agents, in a pharmaceutically acceptable carrier. In some embodiments, the pharmaceutical composition comprising the extracellular vesicle may be administered prior to administration of the additional therapeutic agents. In some embodiments, the pharmaceutical composition comprising the extracellular vesicle may be administered after the administration of the additional therapeutic agents. In some embodiments, the pharmaceutical composition comprising the extracellular vesicle may be administered concurrently with the additional therapeutic agents.

Hereinafter, embodiments of the present disclosure will be described in detail with the following examples. However, the present disclosure is not limited to the examples explained. Rather, the examples are provided to sufficiently transfer the concept of the present disclosure to a person skilled in the art to thorough and complete contents introduced herein.

### EXAMPLE 1: CONSTRUCTION OF PLASMID DNAS

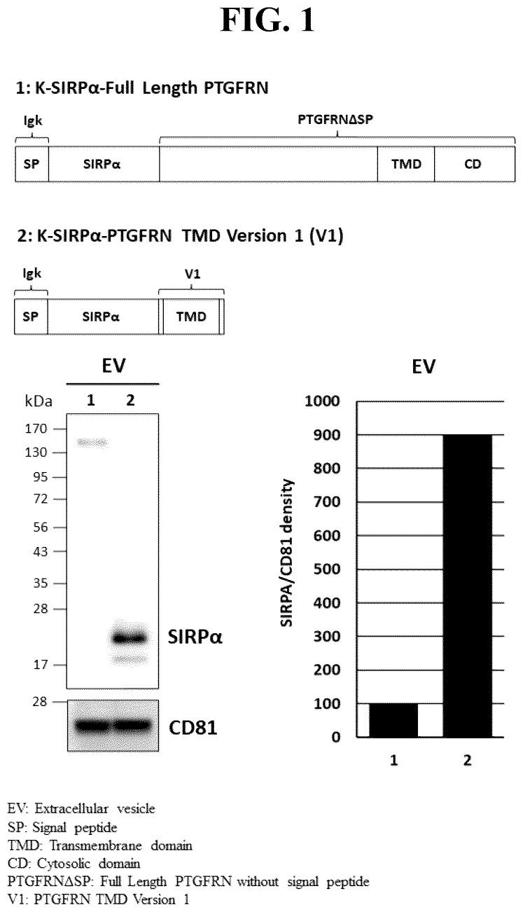

A DNA sequence encoding the Full Length PTGFRN protein and a DNA sequence encoding a mutant SIRP-α protein as, described in U.S. Pat. No. 11,319,360, which is incorporated herein by reference, were fused to prepare a plasmid DNA, K-SIRPα-Full Length PTGFRN vector. A DNA sequence encoding the transmembrane domain (TMD) of the PTGFRN protein and a DNA sequence encoding the mutant SIRP-α protein were fused to prepare plasmid DNAs, K-SIRPα-PTGFRN TMD (V1) vector. See FIG. 1.

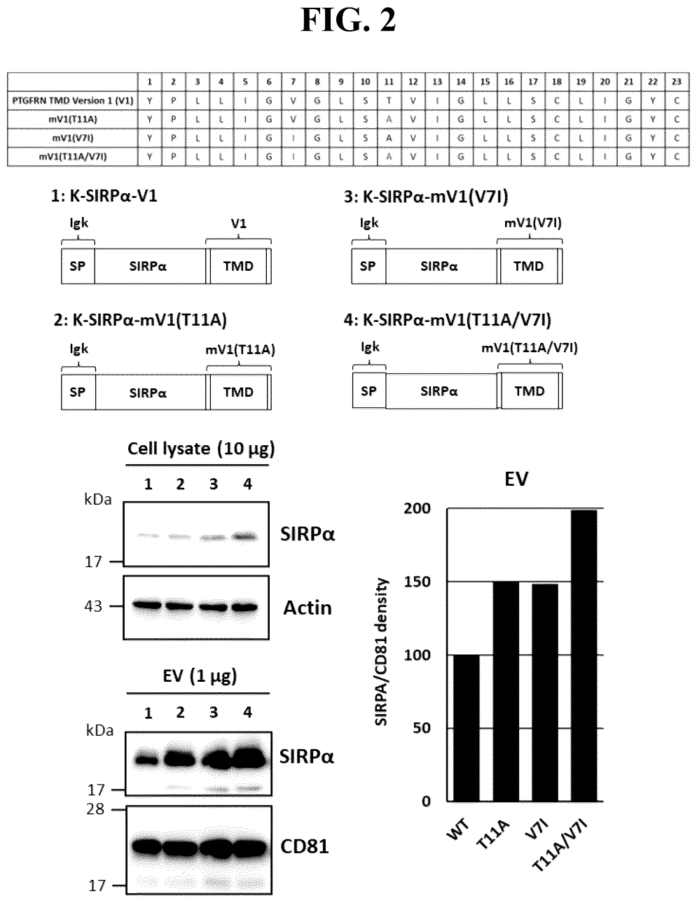

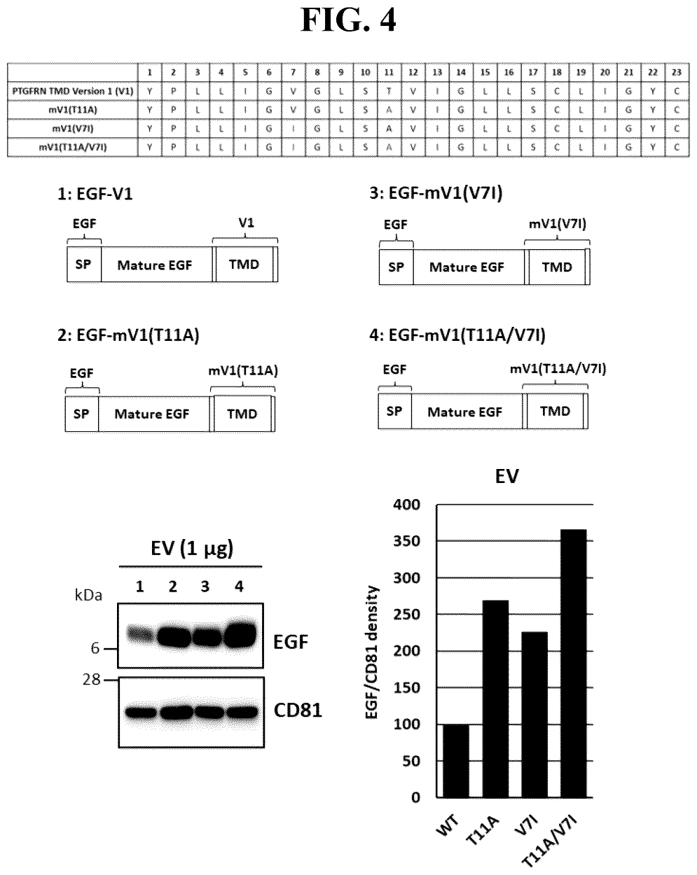

A DNA sequence (i.e., WT) encoding the TMD of the PTGFRN protein and a DNA sequence encoding a therapeutic protein such as a mutant SIRPα and mature form of EGF were prepared. At least one amino acid of the TMD was replaced with another amino acid to prepare additional mutant TMDs. More specifically, an amino acid sequence (i.e., mV1(T11A)) of a variant TMD in which the 11^{th} amino acid T was replaced with amino acid A, an amino acid sequence (i.e., mV1(V7I)) of a variant TMD in which the 7^{th} amino acid V was replaced with amino acid I, an amino acid sequence (i.e., mV1(T11A/V7I)) of a variant TMD in which the 7^{th} amino acid V was replaced with amino acid I, and the 11^{th} amino acid T was replaced with amino acid A were prepared. See FIGS. 2 and 4.

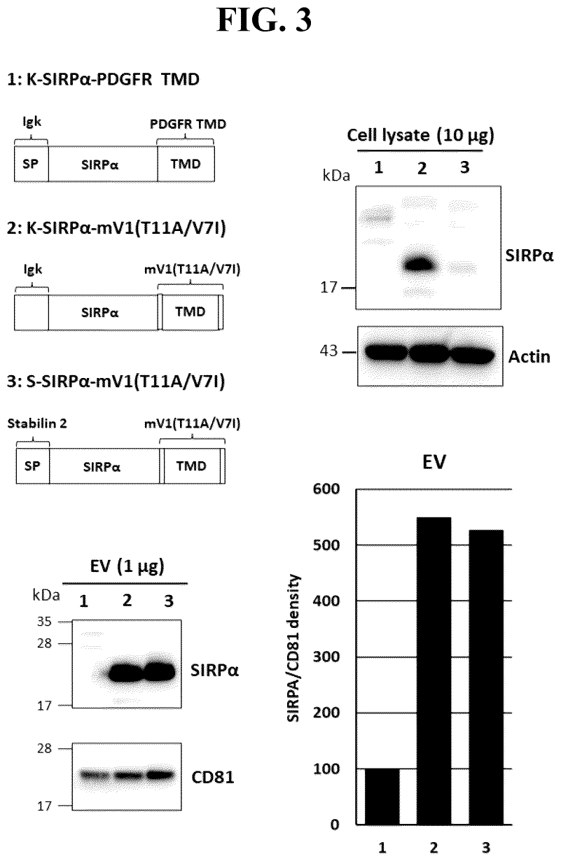

A DNA sequence encoding the variant TMD, K-SIRPα-mV1(T11A/V7I), and a DNA sequence encoding a mutant SIRP-α protein were prepared. A DNA sequence encoding the TMD of the K-SIRPα-mV1(T11A/V7I) plasmid was replaced with the DNA sequence encoding the PDGFR TMD of a commercially available pDisplay vector (Catalog V66020 of Thermo Fisher Scientific) to prepare a plasmid DNA, K-SIRPα-PDGFR TMD vector. A DNA sequence encoding the signal peptide of the K-SIRPα-mV1(T11A/V7I) was replaced with a DNA sequence encoding the signal peptide of stabilin-2 (MMLQHLVIFCLGLVVQNFCSP) from human STAB2[NM_017564] to prepare a plasmid DNA, S-SIRPα-mV1(T11A/V7I) vector. See FIG. 3.

A commercially available DNA sequence encoding the whole EGF protein was used to prepare various plasmid DNAs in accordance with embodiments of the present invention. More specifically, the DNA sequence (RC210817) encoding the whole EGF protein was purchased from Origin, Inc. The DNA sequence encoding the pro-region and the shedding region were removed from the EGF-coding region of the RC210817 vector to prepare a truncated EGF (tEGF) DNA. A DNA sequence encoding the TMD and the CD of the tEGF were replaced with a DNA sequence encoding the PTGFRN TMD (V1) to prepare a plasmid DNA, EGF-V1 vector. A DNA sequence encoding the TMD of the EGF-V1 was replaced with a DNA sequence encoding the mV1(T11A) to prepare a plasmid DNA, EGF-mV1(T11A) vector. A DNA sequence encoding the TMD of the EGF-V1 was replaced with a DNA sequence encoding the mV1(V7I) to prepare a plasmid DNA, EGF-mV1(V7I) vector. A DNA sequence encoding the TMD of the EGF-V1 was replaced with a DNA sequence encoding the mV1(T11A/V7I) to prepare a plasmid DNA, EGF- mV1(T11A/V7I) vector. See FIG. 4.

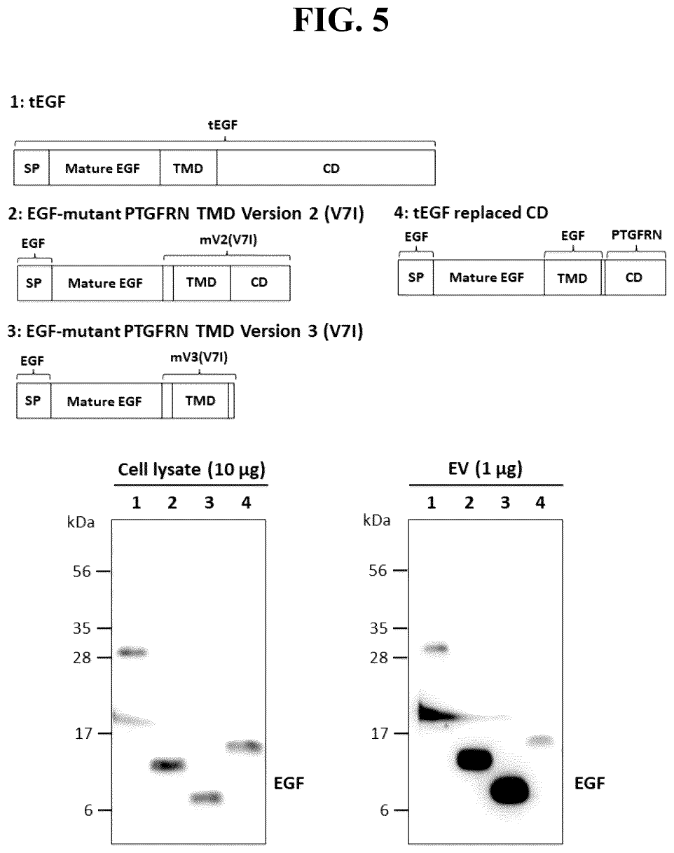

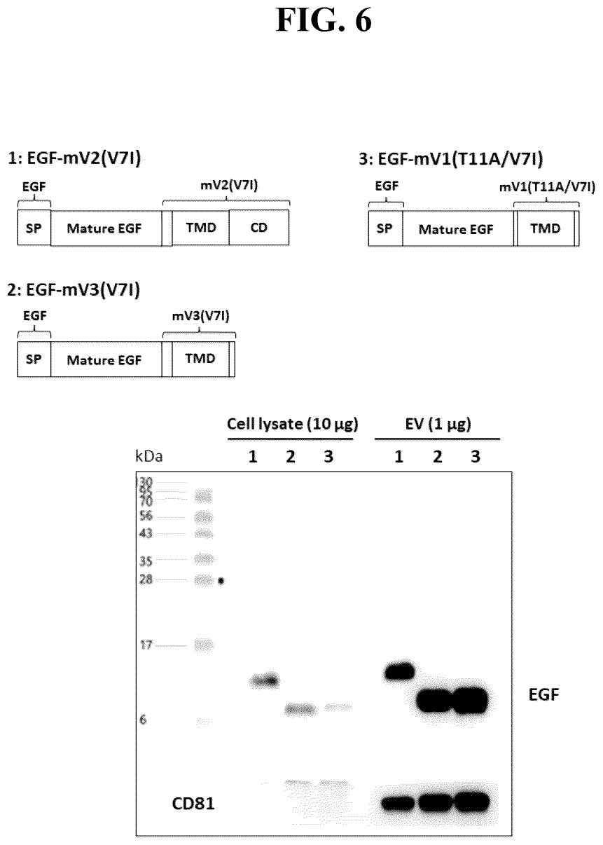

In the PTGFRN TMD Version (V1) sequence, amino acids were added upstream (i.e., DVLNAF) and downstream (i.e., HWCCKKEVQETRRERRRLMSMEMD) to prepare the PTGFRN TMD Version 2 (V2). In the PTGFRN TMD Version (V1) sequence, amino acids were added upstream (i.e., DVLNAF) and downstream (i.e., HWC) to prepare the PTGFRN TMD Version 3 (V3). A DNA sequence encoding the TMD and the CD of the tEGF were replaced to a DNA sequence encoding the mV2(V7I) or mV3(V7I) to prepare a recombinant plasmid DNA, EGF-mV2(V7I) or EGF-mV3(V7I) vector. A DNA sequence encoding the CD of the tEGF plasmid DNA was replaced with a DNA sequence encoding the CD of the PTGFRN protein to prepare a truncated EGF plasmid DNA, tEGF replaced CD vector. See FIGS. 5 and 6.

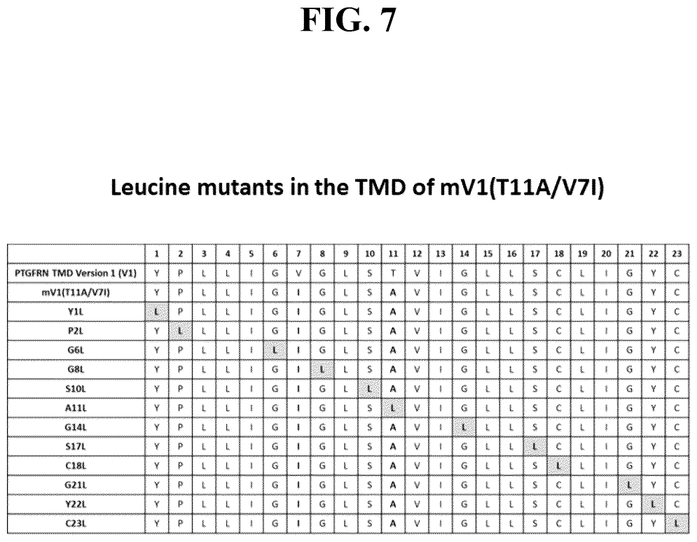

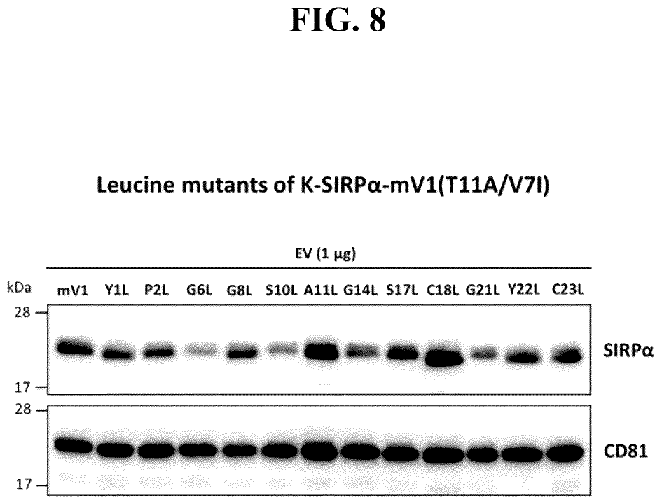

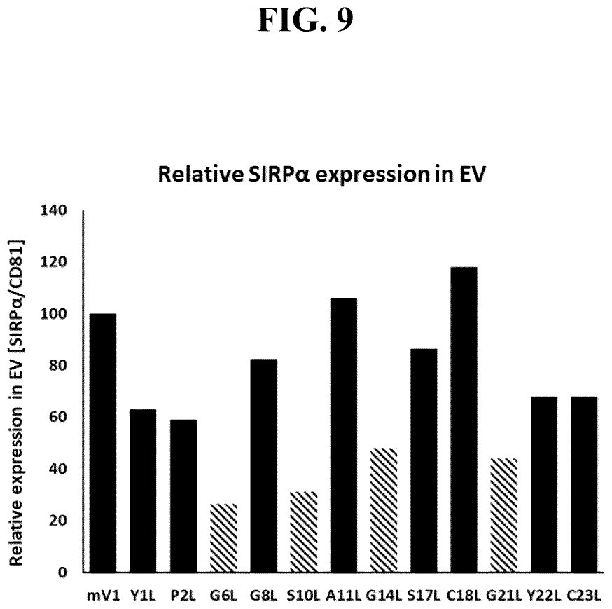

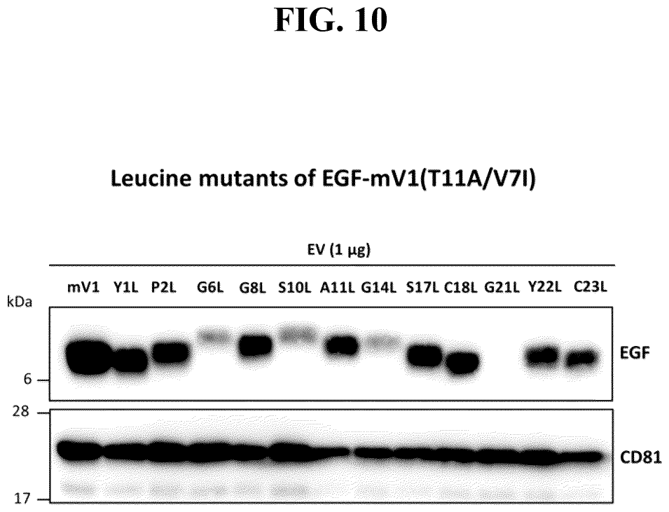

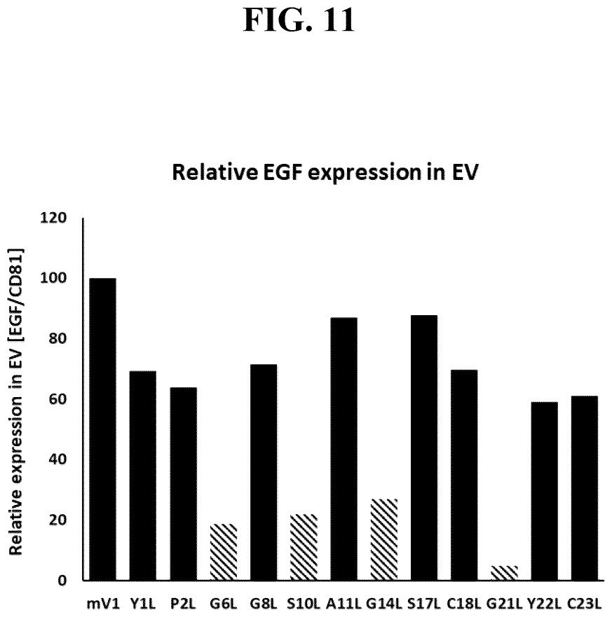

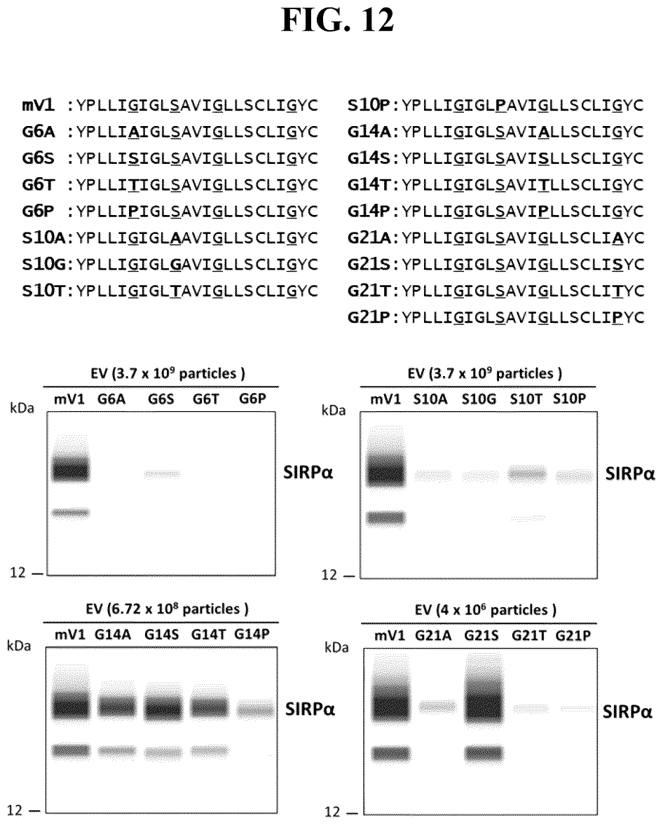

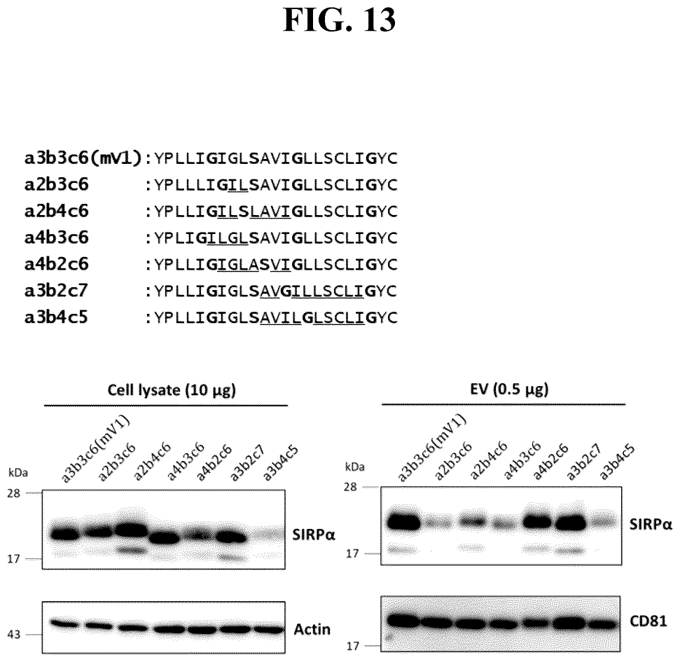

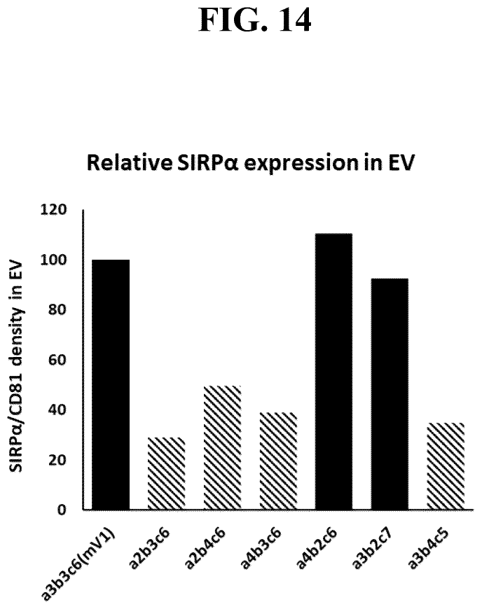

At least one amino acid of the TMD (Extracellular vesicle Sorting Motif, ESM) of the mV1(T11A/V7I) was replaced with another amino acid to prepare additional variant TMDs (FIG. 7-11). Amino acid sequences of variant TMDs (FIG. 12) were prepared by replacing an amino acid of the essential amino acid in ESM encoded by the mV1(T11A/V7I) DNA sequence with another amino acid. Amino acid sequences of variant TMDs (FIG. 13-14) were prepared by deleting or adding one or more amino acids in ESM encoded by the mV1(T11A/V7I) DNA sequence. See FIGS. 7-14.

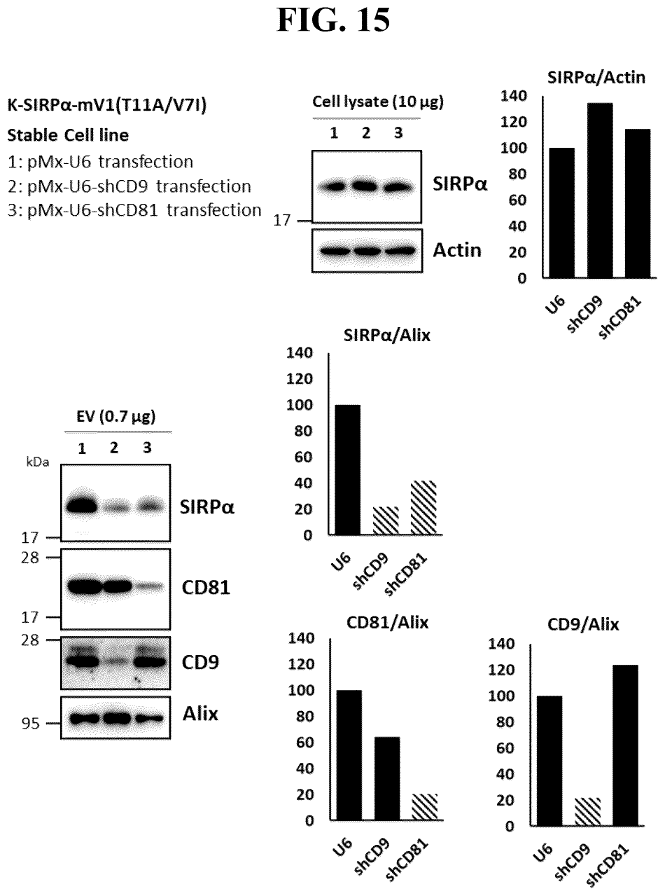

Control plasmid (pMX-U6) or the plasmid encoding CD9 (pMx-U6-shCD9) or CD81 (pMx-U6-shCD81) was prepared to transfect into 293FT cells stably transduced with the K-SIRPα-mV1(T11A/V7I) plasmids. See FIG. 15.

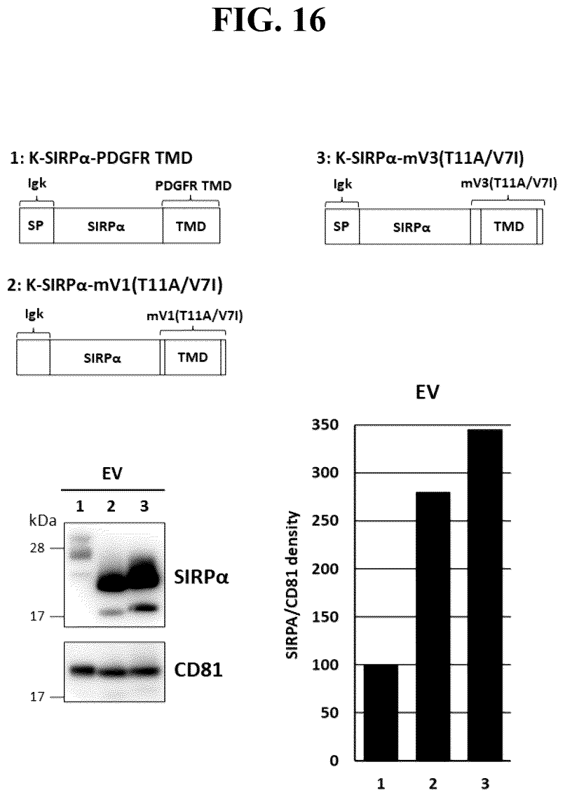

A DNA sequence, K-SIRPα-mV1(T11A/V7I) encoding the variant TMD and a DNA sequence encoding a mutant SIRP-α protein were prepared. A DNA sequence encoding the TMD of the K-SIRPα-mV1(T11A/V7I) was replaced with a DNA sequence encoding the PDGFR TMD of a commercially available pDisplay vector (Catalog V66020 of Thermo Fisher Scientific) to prepare a plasmid DNA, K-SIRPα-PDGFR TMD vector. In the sequence K-SIRPα-mV1(T11A/V7I), sequences upstream (i.e., DVLNAF) and downstream (i.e., HWC) of mV1(T11A/V7I) were added to prepare the K-SIRPα-mV3(T11A/V7I). See FIG. 16.

The above-mentioned plasmids were amplified and isolated according to a protocol of the Qiagen® Plasmid Maxi kit. More specifically, 1 μl (0.1 μg) of the plasmid DNA and 100 μl competent cells DH5α were mixed in a 1.5 ml microcentrifuge tube. Plasmid DNA was introduced to competent cells DH5α by heat shock. To elaborate, the microcentrifuge tube containing the mixture of plasmid DNA and competent cells DH5α was heated at 42° C. for 45 seconds using a heat block. Following this, the heated microcentrifuge tube was placed on ice for 2 minutes. After cooling down, 900 μl antibiotic-free LB agar media was added to the microcentrifuge tube. Then, this microcentrifuge tube was incubated at 37° C. for 45 minutes on a 200-rpm shaker. After incubation, 100 μl from the microcentrifuge tube was spread onto LB media containing plates with 100 μg/ml ampicillin. All plates were incubated overnight at 37° C. On the following day, a colony was taken from the surface of the plate and incubated in 3 ml of LB media with 100 μg/ml ampicillin at 37° C. for 8 hours. After incubation, 1 ml from the mixture of colony and LB media with antibiotics was transferred to a flask containing 500 ml of LB/ampicillin media and incubated overnight at 37° C. The bacterial cells were harvested by centrifugation at 6000×g for 15 min at 4° C. and the bacterial pellet was resuspended in Buffer P1 with RNase A 100 μg/ml. Buffer P2 was added and mixed thoroughly by vigorously inverting the sealed tube 4-6 times, and the resulting mixture was incubated at room temperature for 5 min. Chilled Buffer P3 was added and mixed immediately and thoroughly by vigorously inverting 4-6 times, and the resulting mixture was incubated on ice for 20 min. After centrifuging at 20,000×g for 30 min at 4° C., supernatant containing plasmid DNA was collected promptly. After centrifuging the supernatant again at 20,000×g for 15 min at 4° C., supernatant containing plasmid DNA was collected promptly. After equilibrating a QIAGEN-tip 500 by applying Buffer QBT and allowing the column to empty by gravity flow, the collected supernatant was applied to the QIAGEN-tip and allowed to enter the resin by gravity flow. After washing the QIAGEN-tip with Buffer QC, DNAs were eluted with Buffer QC. The eluted DNAs were precipitated by adding room-temperature isopropanol to the eluted DNA. After mixing and centrifuging immediately at 15,000×g for 30 min at 4° C., the supernatant was carefully decanted. After washing DNA pellet with room-temperature 70% ethanol and centrifuging at ≥15,000×g for 10 min, the supernatant was carefully decanted without disturbing the pellet. After air-drying the pellet for 5-10 min, and the final plasmid DNAs were redissolved in a suitable volume of buffer.

### EXAMPLE 2: ISOLATION OF EXTRACELLULAR VESICLES

HEK293 cells (6×10⁶) were incubated at 37° C. with 5% CO₂ in Dulbecco's modified Eagle's medium (DMEM) to which 10% fetal bovine serum (FBS) was added. At the time when it had 80-90% of confluency, the cells were transfected with a plasmid DNA using transfection agents or infected by a retrovirus for stable cell generation.

In case of transient transfection, the cells were transfected using transfection agents, such as lipofectamine 2000, lipofectamine 3000, or Polyethylenimine (PEI). Cell medium was replaced with DMEM, and mixture of DNA and transfection reagent was added into the cells. The cells were then incubated at 37° C. with 5% CO₂ for 24 hours. 24 hours post transfection, the medium containing transfection agents and plasmids was replaced with DMEM to which 10% FBS and 1% Antibiotic-Antimycotic were added. The transient transfected cells were incubated at 37° C. with 5% CO₂ for 24 hours. 24 hours post recovery, the medium was replaced with DMEM medium to which insulin-transferrin-selenium (Gibco) was added. Serum-free cells were incubated at 37° C. with 5% CO₂ for 48 hours. See, e.g., Gi Kim et al., Xenogenization of tumor cells by fusogenic exosomes in tumor microenvironment ignites and propagates antitumor immunity, SCIENCE ADVANCES, Vol 6, Issue 27 (Jul. 1, 2020), which is incorporated herein by reference.

In case of stable cell generation, Plat-E cells were used to produce retrovirus packaging a retroviral vector containing a DNA sequence of interest and a DNA sequence of puromycin-resistance gene. More particularly, Plat-E cells (2×10⁶) were incubated at 37° C. with 5% CO₂ in Dulbecco's modified Eagle's medium (DMEM) to which 10% FBS was added. At the time when it had 80-90% of confluency, the cells were transfected by the retroviral vector encoding a DNA sequence of interest by using lipofectamine 2000. After 24 hours, culture medium was replaced with DMEM supplemented 10% FBS and incubated for additional 24 hours. When 48 hours passed after transfection was made, culture medium containing viral particles was collected, centrifugated at 3,000 rpm, filtered with 0.45 μm filter, and used for 293FT cell infection. See., e.g., Park, S Y., Yun, Y., Lim, J S. et al. Stabilin-2 modulates the efficiency of myoblast fusion during myogenic differentiation and muscle regeneration. Nat Commun 7, 10871 (2016), which is incorporated herein by reference.

To isolate extracellular vesicles, the supernatants of cells were harvested when 48 hours were passed after the transfection. The supernatants were centrifuged at 300 g for 10 min, 2000 g for 10 min and 10,000 g for 30 min. The supernatants were then filtered and concentrated with a tangential flow filtration (TFF) system or 100 kDa Amicon Ultra-15 centrifugal filter unit. After that, the supernatants were centrifuged at 150,000 g for 3 hours. The extracellular vesicle pellets were resuspended with PBS including s proteinase inhibitor cocktail and preserved at 4° C. See, e.g., Gi Kim et al., Xenogenization of tumor cells by fusogenic exosomes in tumor microenvironment ignites and propagates antitumor immunity, SCIENCE ADVANCES, Vol 6, Issue 27 (Jul. 1, 2020), which is incorporated herein by reference.

### EXAMPLE 3: CHARACTERIZATION OF SURFACE-ENGINEERED EXTRACELLULAR VESICLES

Western Blot test was performed to characterize surface-engineered extracellular vesicles. More specifically, the quantity of whole proteins in extracellular vesicles was measured using the bicinchoninic acid (BCA) protein assay. The standard solution was prepared, and 5 μl of each concentration of bovine serum albumin was applied to the 96-well plate (2, 1, 0.5, 0.25, 0.125 and 0 mg/ml). The extracellular vesicle sample was diluted with PBS, and 5 μl of the resulting sample was applied to the 96 well plate. The reagent A (500113, Bio-Rad) and S (500114, Bio-Rad) were mixed in a ratio of 50 to 1.25 μl of the reagent mixture was applied to the 96-well plate. 200 μl of Reagent B (500115, Bio-Rad) was applied to the 96 well plate and the plate was gently tapped. The samples were incubated 15 min away from the light. The protein amount was measured using Microplate Reader at the wavelength of 750 nm. Additionally, the quantity of extracellular vesicle count was analyzed using a Zetaview. After evaluating the alignment test using QC beads, diluted samples were loaded. 150-200 particles were set to be observed, and then the number of extracellular vesicles was analyzed.

Purified extracellular vesicles were added to RIPA buffer with Protease Inhibitor Cocktail (Calbiochem) to lyse the extracellular vesicles, and they were mixed with an SDS-PAGE sample buffer. The same amount of extracellular vesicle protein was subjected to SDS-PAGE electrophoresis. After gel electrophoresis, bands were transferred to nitrocellulose membranes or methanol activated polyvinylidene difluoride (PVDF) membrane. After being pre-blocked with 5% skim milk which was dissolved in Tween-20-added Tris Buffered Saline (TBST) at room temperature for 1 hour, the membranes were incubated at 4° C. for overnight with the primary antibody. CD81, SIRPα, EGF, and Actin antibody were treated to detect protein expression. The membranes were incubated with the HRP-conjugated secondary antibody, and the blot was then probed using ChemiDoc Imaging System (Bio-Rad) (FIGS. 1-6, 8-10, and 12-15).

Capillary Western Blot test was performed to measure the protein expression. extracellular vesicle samples were prepared by using EZ Standard Pack 1 (ProteinSimple, 96655). Four parts of diluted proteins were combined with one part of 5× Fluorescent Master Mix. Each of the samples was denatured by heat block for 5 minutes at 95° C. 3 μl of the samples were loaded into the appropriate wells of a cartridge. The samples were pre-blocked with Antibody Diluent 2 (ProteinSimple, 95905) for 10 minutes. Appropriate antibodies were diluted to a desired concentration and were used to detect protein expression. The protein expression was detected with Anti-Mouse Secondary Antibody (ProteinSimple, 96113). The samples were analyzed through Compass for SW (ProteinSimple).

As shown in FIG. 1, to evaluate the expression levels of the SIRPα protein, known for its role in facilitating the clearance of pathological cells by phagocytes, utilizing both the entire PTGFRN, which has been reported to exhibit effective protein expression on the EV surface, and a fragment of PTGFRN. The experimental outcomes indicated that the plasmids employing mainly the TMD of PTGFRN, K-SIRPα-PTGFRN TMD Version 1 (V1), demonstrated superior protein expression efficiency compared to the complete PTGFRN, K-SIRPα-Full Length PTGFRN.

K-SIRPα-Full Length PTGFRN
Sequence (SEQ ID NO: 143):
METDTLLLWVLLLWVPGSTGDGSEEELQIIQPDKSVLVAA
GETATLRCTITSLFPVGPIQWERGAGPGRVLIYNQRQGPF
PRVTTVSDTTKRNNMDESIRIGNITPADAGTYYCIKFRKG
SPDDVEFKSGAGTELSVRAKPEFRVVRVPTATLVRVVGTE
LVIPCNVSDYDGPSEQNEDWSFSSLGSSFVELASTWEVG
FPAQLYQERLQRGEILLRRTANDAVELHIKNVQPSDQGHY
KCSTPSTDATVQGNYEDTVQVKVLADSLHVGPSARPPPSL
SLREGEPFELRCTAASASPLHTHLALLWEVHRGPARRSVL
ALTHEGRFHPGLGYEQRYHSGDVRLDTVGSDAYRLSVSRA
LSADQGSYRCIVSEWIAEQGNWQEIQEKAVEVATVVIQPS
VLRAAVPKNVSVAEGKELDLTCNITTDRADDVRPEVTWSF
SRMPDSTLPGSRVLARLDRDSLVHSSPHVALSHVDARSYH
LLVRDVSKENSGYYYCHVSLWAPGHNRSWHKVAEAVSSPA
GVGVTWLEPDYQVYLNASKVPGFADDPTELACRVVDTKSG
EANVRFTVSWYYRMNRRSDNVVTSELLAVMDGDWTLKYGE
RSKQRAQDGDFIFSKEHTDTENFRIQRTTEEDRGNYYCVV
SAWTKQRNNSWVKSKDVFSKPVNIFWALEDSVLVVKARQ
PKPFFAAGNTFEMTCKVSSKNIKSPRYSVLIMAEKPVGD
LSSPNETKYIISLDQDSVVKLENWTDASRVDGVVLEKVQ
EDEFRYRMYQTQVSDAGLYRCMVTAWSPVRGSLWREAAT
SLSNPIEIDFQTSGPIFNASVHSDTPSVIRGDLIKLFCI
ITVEGAALDPDDMAFDVSWFAVHSFGLDKAPVLLSSLDR
KGIVTTSRRDWKSDLSLERVSVLEFLLQVHGSEDQDFGNY
YCSVTPWVKSPTGSWQKEAEIHSKPVFITVKMDVLNAFKY
PLLIGVGLSTVIGLLSCLIGYCSSHWCCKKEVQETRRERR
RLMSMEMD
K-SIRPα-PTGFRN TMD Version 1 (V1)
Sequence (SEQ ID NO: 144):
METDTLLLWVLLLWVPGSTGDGSEEELQIIQPDKSVLVAA
GETATLRCTITSLFPVGPIQWERGAGPGRVLIYNQRQGPF
PRVTTVSDTTKRNNMDFSIRIGNITPADAGTYYCIKFRKG
SPDDVEFKSGAGTELSVRAKPEFKYPLLIGVGLSTVIGLL
SCLIGYCSS

As shown in FIG. 2, to derive motifs with enhanced protein expression efficiency on the EV surface, random mutations of the PTGFRN TMD Version 1 (V1) were used in K-SIRPα-V1. Experimental results demonstrated that the double mutation (K-SIRPα-mV1(T11A/V7I)), in which the 11th amino acid T in V1 was mutated to A, and the 7th amino acid V in V1 was mutated to I, exhibited superior protein expression efficiency compared to the single mutation and the basic wild type PTGFRN TMD (V1).

K-SIRPα-mV1(T11A) Sequence
(SEQ ID NO: 145):
METDTLLLWVLLLWVPGSTGDGSEEELQIIQPDKSVLVAA
GETATLRCTITSLFPVGPIQWERGAGPGRVLIYNQRQGPF
PRVTTVSDTTKRNNMDFSIRIGNITPADAGTYYCIKFRKG
SPDDVEFKSGAGTELSVRAKPEFKYPLLIGVGLSAVIGLL
SCLIGYCSS
K-SIRPα-mV1(V7I) Sequence
(SEQ ID NO: 146):
METDTLLLWVLLLWVPGSTGDGSEEELQIIQPDKSVLVAA
GETATLRCTITSLFPVGPIQWERGAGPGRVLIYNQRQGPF
PRVTTVSDTTKRNNMDESIRIGNITPADAGTYYCIKFRKG
SPDDVEFKSGAGTELSVRAKPEFKYPLLIGIGLSTVIGLL
SCLIGYCSS
K-SIRPα-mV1(T11A/V7I) Sequence
(SEQ ID NO: 147):
METDTLLLWVLLLWVPGSTGDGSEEELQIIQPDKSVLVAA
GETATLRCTITSLFPVGPIQWERGAGPGRVLIYNQRQGPF
PRVTTVSDTTKRNNMDESIRIGNITPADAGTYYCIKFRKG
SPDDVEFKSGAGTELSVRAKPEFKYPLLIGIGLSAVIGLL
SCLIGYCSS

As shown in FIG. 3, to validate the superiority of the derived K-SIRPα-mV1(T11A/V7I), comparative experiments were conducted with PDGFR TMD, which is commonly used for desired protein expression on cell and EV surfaces. The experimental results confirmed that K-SIRPα-mV1(T11A/V7I) demonstrated significantly higher protein expression efficiency on the EV surface compared to K-SIRPα-PDGFR TMD. Furthermore, the difference in protein expression efficiency on the EV surface was examined when the Ig-kappa signal peptide of K-SIRPα-mV1(T11A/V7I) was replaced with the signal peptide of stabilin-2 protein. The results indicated that not only with the Ig-kappa signal peptide but also with the stabilin-2 signal peptide, the mV1(T11A/V7I) maintained the efficient expression of the protein.

K-SIRPα-PDGFR TMD Sequence
(SEQ ID NO: 148):
METDTLLLWVLLLWVPGSTGDYPYDVPDYAGAQPARSMEEE
LQIIQPDKSVLVAAGETATLRCTITSLFPVGPIQWFRGAG
PGRVLIYNQRQGPFPRVTTVSDTTKRNNMDESIRIGNITP
ADAGTYYCIKFRKGSPDDVEFKSGAGTELSVRAKPVDEQK
LISEEDLNAVGODTQEVIVVPHSLPFKVVVISAILALVVL
TIISLIILIMLWQKKPR
S-SIRPα-mV1(T11A/V7I) Sequence
(SEQ ID NO: 149):
MMLQHLVIFCLGLVVQNFCSPGSEEELQIIQPDKSVLVAA
GETATLRCTITSLFPVGPIQWERGAGPGRVLIYNQRQGPF
PRVTTVSDTTKRNNMDFSIRIGNITPADAGTYYCIKFRKG
SPDDVEFKSGAGTELSVRAKPEFKYPLLIGIGLSAVIGLL
SCLIGYCSS

As shown in FIG. 4, to demonstrate the versatility of the motif, the fusion of mature EGF protein, a regenerative factor, with mV1(T11A/V7I) instead of the SIRPα protein was evaluated. The experimental results showed that, similar to SIRPα, EGF also exhibited superior protein expression efficiency when the 11th amino acid T in V1 was mutated to A, and the 7th amino acid V in V1 was mutated to I. The double mutation EGF-mV1(T11A/V7I) exhibited enhanced protein expression efficiency compared to the single mutation and the basic wild type PTGFRN TMD (V1).

EGF-V1 Sequence
(SEQ ID NO: 150):
MLLTLIILLPVVSKFSFVSLSANSDSECPLSHDGYCLHDG
VCMYIEALDKYACNCVVGYIGERCQYRDLKWWELREFKYP
LLIGVGLSTVIGLLSCLIGYCSS
EGF-mV1(T11A) Sequence
(SEQ ID NO: 151):
MLLTLIILLPVVSKFSFVSLSANSDSECPLSHDGYCLHDG
VCMYIEALDKYACNCVVGYIGERCQYRDLKWWELREFKYP
LLIGVGLSAVIGLLSCLIGYCSS
EGF-mV1(V7I) Sequence
(SEQ ID NO: 152):
MLLTLIILLPVVSKFSFVSLSANSDSECPLSHDGYCLHDG
VCMYIEALDKYACNCVVGYIGERCQYRDLKWWELREFKYP
LLIGIGLSTVIGLLSCLIGYCSS
EGF-mV1(T11A/V7I) Sequence
(SEQ ID NO: 153):
MLLTLIILLPVVSKFSFVSLSANSDSECPLSHDGYCLHDG
VCMYIEALDKYACNCVVGYIGERCQYRDLKWWELREFKYP
LLIGIGLSAVIGLLSCLIGYCSS

As shown in FIG. 5, the EGF expression of EGF-mutant PTGFRN TMD Version 2 (V7I) was found to be higher than that of tEGF, and the EGF expression of EGF-mutant PTGFRN TMD Version 3 (V7I) was similar to or greater than that of EGF-mV2(V7I). Conversely, both tEGF replaced CD which lacked the PTGFRN TMD variant, demonstrated very low EGF expression efficiency on the EV surface. These findings suggest that the TMD of the PTGFRN protein plays a crucial role in displaying therapeutic proteins on the surface of EVs.

tEGF Sequence (SEQ ID NO: 154):
MLLTLIILLPVVSKFSFVSLSANSDSECPLSHDGYCLHDG
VCMYIEALDKYACNCVVGYIGERCQYRDLKWWELRVIVVA
VCVVVLVMLLLLSLWGAHYYRTQKLLSKNPKNPYEESSRD
VRSRRPADTEDGMSSCPQPWFVVIKEHQDLKNGGQPVAGE
DGQAADGSMQPTSWRQEPQLCGMGTEQGCWIPVSSDKGSC
PQVMERSFHMPSYGTQTLEGGVEKPHSLLSANPLWQQRAL
DPPHQMELTQ
EGF-mutant PTGFRN TMD Version 2 (V7I)
Sequence (SEQ ID NO: 155):
MLLTLIILLPVVSKFSFVSLSANSDSECPLSHDGYCLHDG
VCMYIEALDKYACNCVVGYIGERCQYRDLKWWELREFDVL
NAFKYPLLIGIGLSTVIGLLSCLIGYCSSHWCCKKEVQET
RRERRRLMSMEMD
EGF-mutant PTGFRN TMD Version 3 (V7I)
Sequence (SEQ ID NO: 156):
MLLTLIILLPVVSKFSFVSLSANSDSECPLSHDGYCLHDG
VCMYIEALDKYACNCVVGYIGERCQYRDLKWWELREFDVL
NAFKYPLLIGIGLSTVIGLLSCLIGYCSSHWC
tEGF replaced CD Sequence
(SEQ ID NO: 157):
MLLTLIILLPVVSKFSFVSLSANSDSECPLSHDGYCLHDG
VCMYIEALDKYACNCVVGYIGERCQYRDLKWWELRVIVVA
VCVVVLVMLLLLSLWGAHYYRTQEFHWCCKKEVQETRRER
RRLMSMEMD

As shown in FIG. 6, it was observed that the EGF expression of EGF-mV1(T11A/V7I) and EGF-mV3(V7I) was higher than that of EGF-mV2(V7I) which contained both the TMD and CD of the PTGFRN protein. This finding suggests that the CD of the PTGFRN protein is not necessary for EV sorting or targeting.

As depicted in FIGS. 7-11, to determine the critical sequence for the desired protein's EV surface expression in the variant TMD (ESM, derived from mV1(T11A/V7I)), plasmids were generated by conducting single mutations in ESM to L and aimed to derive the important TMD amino acid sequence pattern for the introduced protein's EV sorting (FIG. 7). It was identified that the SIRPα or EGF protein expression of the tested DNAs was notably decreased when the 6th amino acid G, the 10th amino acid S, the 14th amino acid G, the 21st amino acid G, or any combination thereof was/were replaced with other amino acid(s). The results indicate that the G-S-G-G pattern is essential for protein EV sorting.

As shown in FIG. 12, to verify whether the critical amino acids in the ESM, specifically the 6th amino acid G, the 10th amino acid S, the 14th amino acid G, and the 21st amino acid G, can be replaced with other amino acids besides L, the EV sorting efficacy of the introduced protein by changing each critical amino acid to four different amino acids was evaluated. The experimental results reveal that G can be present at the 6th amino acid position, S at the 10th amino acid position, G, A, S, or T at the 14th amino acid position, and G or S at the 21st amino acid position.

As depicted in FIGS. 13-14. to evaluate the possible number of amino acids can be presented between the critical amino acids in the derived G-S-G-G sequence, the EV sorting efficacy of proteins was assessed. The number of amino acids between the 6th G and the 10th S was designated as ‘a’, the number of amino acids between the 10th S and the 14th G was ‘b’, and the number of amino acids between the 14th G and the 21st G was ‘c’. Six different plasmids with varying numbers of a, b, and c amino acids were generated compared to the original sequence, and the EV sorting efficacy of the proteins was evaluated. The experimental results reveal that it is possible to have 3-4 amino acids for ‘a’, 2-3 amino acids for ‘b’, and 6-7 amino acids for ‘c’.

As shown in FIG. 15, it was identified that the SIRPα expression on EVs obtained from the DNA constructs in accordance with embodiments of the present invention (K-SIRPα-mV1(T11A/V7I)) was significantly decreased when transfected with CD9 or CD81 shRNA. These results suggest that CD9 and CD81 proteins are associated with the EV surface expression mechanism of the proteins introduced by the ESM.

As shown in FIG. 16, the protein EV sorting efficiency according to the addition of a few amino acids before and after the mV1(T11A/V7I) while possessing ESM was compared. As a result of the experiments, both mV1(T11A/V7I) and mV3(T11A/V7I) showed excellent SIRPα protein expression efficiency on the EV surface, but mV3(T11A/V7I) demonstrated a slightly higher EV sorting efficiency.

K-SIRPα-mV3(T11A/V7I) Sequence
(SEQ ID NO: 158):
METDTLLLWVLLLWVPGSTGDGSEEELQIIQPDKSVLVAA
GETATLRCTITSLFPVGPIQWERGAGPGRVLIYNQRQGPF
PRVTTVSDTTKRNNMDFSIRIGNITPADAGTYYCIKFRKG
SPDDVEFKSGAGTELSVRAKPEFDVLNAFKYPLLIGIGLS
AVIGLLSCLIGYCSSHWC

While the present disclosure has been described herein in connection with certain embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus, the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure.

Changes may be made in the formulation of the various compositions described herein, the methods described herein or in the steps or the sequence of steps of the methods described herein without departing from the spirit and scope of the present disclosure. Further, while various embodiments of the present disclosure have been described in the claims herein below, it is not intended that the present disclosure be limited to these particular claims.

What is claimed is:

1. A DNA construct comprising a DNA sequence encoding a scaffold peptide, wherein the amino acid sequence of the scaffold peptide includes a sequence represented by G-a-S-b-X1-c-X2, in which:
X1 represents G, A, S, or T;
X2 represents G or S;
a represents 3-4 amino acids;
b represents 2-3 amino acids;
c represents 6-7 amino acids;
G represents glycine;
S represents serine;
A represents alanine; and
T represents threonine.

2. The DNA construct of claim 1, wherein the sequence G-a-S-b-X1-c-X2 has 15-17 amino acids.

3. The DNA construct of claim 1, wherein the scaffold peptide has 22-57 amino acids.

4. The DNA construct of claim 1, wherein the a, b, and c includes V, G, L, I, A, T, S, C, F, W, Y, and P, in which V represents valine, G represents glycine, L represents leucine, I represents isoleucine, A represents alanine, T represents threonine, S represents serine, C represents cysteine, F represents phenylalanine, W represents tryptophan, Y represents tyrosine, and P represents proline.

5. The DNA construct of claim 1, wherein a represents 3-4 amino acids selected from the group consisting of V, G, L, I, T and A, in which V represents valine, G represents glycine, L represents leucine, I represents isoleucine, T represents threonine and A represents alanine.

6. The DNA construct of claim 5, wherein a represents VGL, IGL, VGLT, IGLT, VGLA, or IGLA.

7. The DNA construct of claim 1, wherein b represents 2-3 amino acids selected from the group consisting of V, I, A, and T, in which V represents valine, I represents isoleucine, A represents alanine, and T represents threonine.

8. The DNA construct of claim 7, wherein b represents VI, AV, TVI, or AVI.

9. The DNA construct of claim 1, wherein c represents 6-7 amino acids selected from the group consisting of L, S, C, and I, in which L represents leucine, S represents serine, C represents cysteine, and I represents isoleucine.

10. The DNA construct of claim 9, wherein c represents LLSCLI or ILLSCLI.

11. The DNA construct of claim 1, wherein the sequence G-a-S-b-X1-c-X2 is any one of ESM SEQ ID NOS: 1-100.

12. The DNA construct of claim 1, wherein the scaffold peptide further comprises KYPLLI at the N-terminal of the sequence G-a-S-b-X1-c-X2, in which K represents lysine, Y represents tyrosine, P represents proline, L represents leucine, and I represents isoleucine.

13. The DNA construct of claim 1, wherein the scaffold peptide further comprises DVLNAFKYPLLI at the N-terminal of the sequence G-a-S-b-X1-c-X2, in which D represents aspartic acid, V represents valine, L represents leucine, N represents asparagine, A represents alanine, F represents phenylalanine, K represents lysine, Y represents tyrosine, P represents proline, L represents leucine, and I represents isoleucine.

14. The DNA construct of claim 1, wherein the scaffold peptide further comprises YCSS at the C-terminal of the sequence G-a-S-b-X1-c-X2, in which Y represents tyrosine, and C represents cysteine, and S represents serine. The DNA construct of claim 1, wherein the scaffold peptide further comprises YCSSHWC at the C-terminal of the sequence G-a-S-b-X1-c-X2, in which Y represents tyrosine, C represents cysteine, S represents serine, H represents histidine, and W represents tryptophan.

16. The DNA construct of claim 1, which further comprises a DNA sequence encoding an amino acid sequence of a target protein.

17. The DNA construct of claim 16, wherein the target protein is a therapeutic protein.

18. A vector comprising the DNA construct of claim 1.

19. A host cell comprising the vector of claim 18.

20. An extracellular vesicle isolated from the host cell of claim 19, wherein the scaffold peptide is present at a desired position of the extracellular vesicle.

21. An extracellular vesicle comprising the scaffold peptide encoded by the DNA construct according to claim 1.

22. The extracellular vesicle of claim 20 or 21, wherein another extracellular peptide comprises CD9, CD63, CD81, PDGFR, PTGFRN, GPI anchor proteins, lactadherin, syndecan, synaptotagmin, apoptosis-linked gene 2-interacting protein X (ALIX), syntenin, LAMP2, LAMP2B, a fragment or variant thereof, a variant of the fragment, and a fragment of the variant.

23. The extracellular vesicle of claim 20 or 21, which further comprises a target protein.

24. The extracellular vesicle of claim 23, wherein the target protein is a therapeutic protein.

25. The extracellular vesicle of claim 23, wherein the scaffold peptide is fused to the target protein.

26. The extracellular vesicle of claim 20 or 21, wherein the scaffold peptide comprises an affinity tag having affinity to a binding agent.

27. The extracellular vesicle of claim 20 or 21, wherein the scaffold peptide further comprises a targeting moiety.

28. The extracellular vesicle of claim 20 or 21, wherein the extracellular vesicle further comprises a therapeutic substance.

29. The extracellular vesicle of claim 28, wherein the therapeutic substance is selected from the group consisting of a nucleotide, an amino acid, a lipid, a carbohydrate, a small molecule, and any combination thereof.

30. The extracellular vesicle of claim 28, wherein the therapeutic substance is fused to the scaffold peptide and/or is encapsulated in the extracellular vesicle.

31. A pharmaceutical composition comprising the extracellular vesicle of claim 20 or 21, and a pharmaceutically acceptable carrier.

32. A method for preventing, ameliorating, or treating disease, disorder, or condition associated with nervous, digestive, endocrine, skeletal, respiratory, integumentary, lymphatic, reproductive, muscular, excretory, or immune system, the method comprising administering to a subject in need a therapeutically effective amount of the pharmaceutical composition of claim 31.


SEQUENCE LISTING (158 sequences)

SEQ ID NO: 1 - protein, 16 aa, synthetic construct
GVGLSTVIGLLSCLIG

SEQ ID NO: 2 - protein, 16 aa, synthetic construct
GIGLSTVIGLLSCLIG

SEQ ID NO: 3 - protein, 16 aa, synthetic construct
GVGLSAVIGLLSCLIG

SEQ ID NO: 4 - protein, 16 aa, synthetic construct
GIGLSAVIGLLSCLIG

SEQ ID NO: 5 - protein, 16 aa, synthetic construct
GILLSAVIGLLSCLIG

SEQ ID NO: 6 - protein, 16 aa, synthetic construct
GIGLSLVIGLLSCLIG

SEQ ID NO: 7 - protein, 16 aa, synthetic construct
GIGLSAVIGLLLCLIG

SEQ ID NO: 8 - protein, 16 aa, synthetic construct
GIGLSAVIGLLSLLIG

SEQ ID NO: 9 - protein, 16 aa, synthetic construct
GIGLSAVIALLSCLIG

SEQ ID NO: 10 - protein, 16 aa, synthetic construct
GIGLSAVISLLSCLIG

SEQ ID NO: 11 - protein, 16 aa, synthetic construct
GIGLSAVITLLSCLIG

SEQ ID NO: 12 - protein, 16 aa, synthetic construct
GIGLSAVIGLLSCLIS

SEQ ID NO: 13 - protein, 16 aa, synthetic construct
GIGLASVIGLLSCLIG

SEQ ID NO: 14 - protein, 16 aa, synthetic construct
GIGLSAVGILLSCLIG

SEQ ID NO: 15 - protein, 16 aa, synthetic construct
GVGLSTVIALLSCLIG

SEQ ID NO: 16 - protein, 16 aa, synthetic construct
GVGLSTVISLLSCLIG

SEQ ID NO: 17 - protein, 16 aa, synthetic construct
GVGLSTVITLLSCLIG

SEQ ID NO: 18 - protein, 16 aa, synthetic construct
GVGLSTVIGLLSCLIS

SEQ ID NO: 19 - protein, 16 aa, synthetic construct
GVGLSTVIALLSCLIS

SEQ ID NO: 20 - protein, 16 aa, synthetic construct
GVGLSTVISLLSCLIS

SEQ ID NO: 21 - protein, 16 aa, synthetic construct
GVGLSTVITLLSCLIS

SEQ ID NO: 22 - protein, 16 aa, synthetic construct
GIGLSTVIALLSCLIG

SEQ ID NO: 23 - protein, 16 aa, synthetic construct
GIGLSTVISLLSCLIG

SEQ ID NO: 24 - protein, 16 aa, synthetic construct
GIGLSTVITLLSCLIG

SEQ ID NO: 25 - protein, 16 aa, synthetic construct
GIGLSTVIALLSCLIS

SEQ ID NO: 26 - protein, 16 aa, synthetic construct
GIGLSTVISLLSCLIS

SEQ ID NO: 27 - protein, 16 aa, synthetic construct
GIGLSTVITLLSCLIS

SEQ ID NO: 28 - protein, 16 aa, synthetic construct
GVGLSAVIALLSCLIG

SEQ ID NO: 29 - protein, 16 aa, synthetic construct
GVGLSAVISLLSCLIG

SEQ ID NO: 30 - protein, 16 aa, synthetic construct
GVGLSAVITLLSCLIG

SEQ ID NO: 31 - protein, 16 aa, synthetic construct
GVGLSAVIGLLSCLIS

SEQ ID NO: 32 - protein, 16 aa, synthetic construct
GVGLSAVIALLSCLIS

SEQ ID NO: 33 - protein, 16 aa, synthetic construct
GVGLSAVISLLSCLIS

SEQ ID NO: 34 - protein, 16 aa, synthetic construct
GVGLSAVITLLSCLIS

SEQ ID NO: 35 - protein, 16 aa, synthetic construct
GILLSAVIALLSCLIG

SEQ ID NO: 36 - protein, 16 aa, synthetic construct
GILLSAVISLLSCLIG

SEQ ID NO: 37 - protein, 16 aa, synthetic construct
GILLSAVITLLSCLIG

SEQ ID NO: 38 - protein, 16 aa, synthetic construct
GILLSAVIGLLSCLIS

SEQ ID NO: 39 - protein, 16 aa, synthetic construct
GILLSAVIALLSCLIS

SEQ ID NO: 40 - protein, 16 aa, synthetic construct
GILLSAVISLLSCLIS

SEQ ID NO: 41 - protein, 16 aa, synthetic construct
GILLSAVITLLSCLIS

SEQ ID NO: 42 - protein, 16 aa, synthetic construct
GIGLSLVIALLSCLIG

SEQ ID NO: 43 - protein, 16 aa, synthetic construct
GIGLSLVISLLSCLIG

SEQ ID NO: 44 - protein, 16 aa, synthetic construct
GIGLSLVITLLSCLIG

SEQ ID NO: 45 - protein, 16 aa, synthetic construct
GIGLSLVIGLLSCLIS

SEQ ID NO: 46 - protein, 16 aa, synthetic construct
GIGLSLVIALLSCLIS

SEQ ID NO: 47 - protein, 16 aa, synthetic construct
GIGLSLVISLLSCLIS

SEQ ID NO: 48 - protein, 16 aa, synthetic construct
GIGLSLVITLLSCLIS

SEQ ID NO: 49 - protein, 16 aa, synthetic construct
GIGLSAVIALLLCLIG

SEQ ID NO: 50 - protein, 16 aa, synthetic construct
GIGLSAVISLLLCLIG

SEQ ID NO: 51 - protein, 16 aa, synthetic construct
GIGLSAVITLLLCLIG

SEQ ID NO: 52 - protein, 16 aa, synthetic construct
GIGLSAVIGLLLCLIS

SEQ ID NO: 53 - protein, 16 aa, synthetic construct
GIGLSAVIALLLCLIS

SEQ ID NO: 54 - protein, 16 aa, synthetic construct
GIGLSAVISLLLCLIS

SEQ ID NO: 55 - protein, 16 aa, synthetic construct
GIGLSAVITLLLCLIS

SEQ ID NO: 56 - protein, 16 aa, synthetic construct
GIGLSAVIALLSLLIG

SEQ ID NO: 57 - protein, 16 aa, synthetic construct
GIGLSAVISLLSLLIG

SEQ ID NO: 58 - protein, 16 aa, synthetic construct
GIGLSAVITLLSLLIG

SEQ ID NO: 59 - protein, 16 aa, synthetic construct
GIGLSAVIGLLSLLIS

SEQ ID NO: 60 - protein, 16 aa, synthetic construct
GIGLSAVIALLSLLIS

SEQ ID NO: 61 - protein, 16 aa, synthetic construct
GIGLSAVISLLSLLIS

SEQ ID NO: 62 - protein, 16 aa, synthetic construct
GIGLSAVITLLSLLIS

SEQ ID NO: 63 - protein, 16 aa, synthetic construct
GIGLASVIALLSCLIG

SEQ ID NO: 64 - protein, 16 aa, synthetic construct
GIGLASVISLLSCLIG

SEQ ID NO: 65 - protein, 16 aa, synthetic construct
GIGLASVITLLSCLIG

SEQ ID NO: 66 - protein, 16 aa, synthetic construct
GIGLASVIGLLSCLIS

SEQ ID NO: 67 - protein, 16 aa, synthetic construct
GIGLASVIALLSCLIS

SEQ ID NO: 68 - protein, 16 aa, synthetic construct
GIGLASVISLLSCLIS

SEQ ID NO: 69 - protein, 16 aa, synthetic construct
GIGLASVITLLSCLIS

SEQ ID NO: 70 - protein, 16 aa, synthetic construct
GIGLSAVAILLSCLIG

SEQ ID NO: 71 - protein, 16 aa, synthetic construct
GIGLSAVSILLSCLIG

SEQ ID NO: 72 - protein, 16 aa, synthetic construct
GIGLSAVTILLSCLIG

SEQ ID NO: 73 - protein, 16 aa, synthetic construct
GIGLSAVGILLSCLIS

SEQ ID NO: 74 - protein, 16 aa, synthetic construct
GIGLSAVAILLSCLIS

SEQ ID NO: 75 - protein, 16 aa, synthetic construct
GIGLSAVSILLSCLIS

SEQ ID NO: 76 - protein, 16 aa, synthetic construct
GIGLSAVTILLSCLIS

SEQ ID NO: 77 - protein, 17 aa, synthetic construct
GIGLASAVIGLLSCLIG

SEQ ID NO: 78 - protein, 17 aa, synthetic construct
GIGLASAVIALLSCLIG

SEQ ID NO: 79 - protein, 17 aa, synthetic construct
GIGLASAVISLLSCLIG

SEQ ID NO: 80 - protein, 17 aa, synthetic construct
GIGLASAVITLLSCLIG

SEQ ID NO: 81 - protein, 17 aa, synthetic construct
GIGLASAVIGLLSCLIS

SEQ ID NO: 82 - protein, 17 aa, synthetic construct
GIGLASAVIALLSCLIS

SEQ ID NO: 83 - protein, 17 aa, synthetic construct
GIGLASAVISLLSCLIS

SEQ ID NO: 84 - protein, 17 aa, synthetic construct
GIGLASAVITLLSCLIS

SEQ ID NO: 85 - protein, 17 aa, synthetic construct
GIGLSAVIGILLSCLIG

SEQ ID NO: 86 - protein, 17 aa, synthetic construct
GIGLSAVIAILLSCLIG

SEQ ID NO: 87 - protein, 17 aa, synthetic construct
GIGLSAVISILLSCLIG

SEQ ID NO: 88 - protein, 17 aa, synthetic construct
GIGLSAVITILLSCLIG

SEQ ID NO: 89 - protein, 17 aa, synthetic construct
GIGLSAVIGILLSCLIS

SEQ ID NO: 90 - protein, 17 aa, synthetic construct
GIGLSAVIAILLSCLIS

SEQ ID NO: 91 - protein, 17 aa, synthetic construct
GIGLSAVISILLSCLIS

SEQ ID NO: 92 - protein, 17 aa, synthetic construct
GIGLSAVITILLSCLIS

SEQ ID NO: 93 - protein, 18 aa, synthetic construct
GIGLASAVIGILLSCLIG

SEQ ID NO: 94 - protein, 18 aa, synthetic construct
GIGLASAVIAILLSCLIG

SEQ ID NO: 95 - protein, 18 aa, synthetic construct
GIGLASAVISILLSCLIG

SEQ ID NO: 96 - protein, 18 aa, synthetic construct
GIGLASAVITILLSCLIG

SEQ ID NO: 97 - protein, 18 aa, synthetic construct
GIGLASAVIGILLSCLIS

SEQ ID NO: 98 - protein, 18 aa, synthetic construct
GIGLASAVIAILLSCLIS

SEQ ID NO: 99 - protein, 18 aa, synthetic construct
GIGLASAVISILLSCLIG

SEQ ID NO: 100 - protein, 18 aa, synthetic construct
GIGLASAVITILLSCLIG

SEQ ID NO: 101 - protein, 26 aa, synthetic construct
KYPLLIGVGLSTVIGLLSCLIGYCSS

SEQ ID NO: 102 - protein, 26 aa, synthetic construct
KYPLLIGIGLSTVIGLLSCLIGYCSS

SEQ ID NO: 103 - protein, 26 aa, synthetic construct
KYPLLIGVGLSAVIGLLSCLIGYCSS

SEQ ID NO: 104 - protein, 26 aa, synthetic construct
KYPLLIGIGLSAVIGLLSCLIGYCSS

SEQ ID NO: 105 - protein, 26 aa, synthetic construct
KYPLLIGILLSAVIGLLSCLIGYCSS

SEQ ID NO: 106 - protein, 26 aa, synthetic construct
KYPLLIGIGLSLVIGLLSCLIGYCSS

SEQ ID NO: 107 - protein, 26 aa, synthetic construct
KYPLLIGIGLSAVIGLLLCLIGYCSS

SEQ ID NO: 108 - protein, 26 aa, synthetic construct
KYPLLIGIGLSAVIGLLSLLIGYCSS

SEQ ID NO: 109 - protein, 26 aa, synthetic construct
KYPLLIGIGLSAVIALLSCLIGYCSS

SEQ ID NO: 110 - protein, 26 aa, synthetic construct
KYPLLIGIGLSAVISLLSCLIGYCSS

SEQ ID NO: 111 - protein, 26 aa, synthetic construct
KYPLLIGIGLSAVITLLSCLIGYCSS

SEQ ID NO: 112 - protein, 26 aa, synthetic construct
KYPLLIGIGLSAVIGLLSCLISYCSS

SEQ ID NO: 113 - protein, 26 aa, synthetic construct
KYPLLIGIGLASVIGLLSCLIGYCSS

SEQ ID NO: 114 - protein, 26 aa, synthetic construct
KYPLLIGIGLSAVGILLSCLIGYCSS

SEQ ID NO: 115 - protein, 56 aa, synthetic construct
DVLNAFKYPLLIGVGLSTVIGLLSCLIGYCSSHWCCKKEVQETRRERRRLMSMEMD

SEQ ID NO: 116 - protein, 56 aa, synthetic construct
DVLNAFKYPLLIGIGLSTVIGLLSCLIGYCSSHWCCKKEVQETRRERRRLMSMEMD

SEQ ID NO: 117 - protein, 56 aa, synthetic construct
DVLNAFKYPLLIGVGLSAVIGLLSCLIGYCSSHWCCKKEVQETRRERRRLMSMEMD

SEQ ID NO: 118 - protein, 56 aa, synthetic construct
DVLNAFKYPLLIGIGLSAVIGLLSCLIGYCSSHWCCKKEVQETRRERRRLMSMEMD

SEQ ID NO: 119 - protein, 56 aa, synthetic construct
DVLNAFKYPLLIGILLSAVIGLLSCLIGYCSSHWCCKKEVQETRRERRRLMSMEMD

SEQ ID NO: 120 - protein, 56 aa, synthetic construct
DVLNAFKYPLLIGIGLSLVIGLLSCLIGYCSSHWCCKKEVQETRRERRRLMSMEMD

SEQ ID NO: 121 - protein, 56 aa, synthetic construct
DVLNAFKYPLLIGIGLSAVIGLLLCLIGYCSSHWCCKKEVQETRRERRRLMSMEMD

SEQ ID NO: 122 - protein, 56 aa, synthetic construct
DVLNAFKYPLLIGIGLSAVIGLLSLLIGYCSSHWCCKKEVQETRRERRRLMSMEMD

SEQ ID NO: 123 - protein, 56 aa, synthetic construct
DVLNAFKYPLLIGIGLSAVIALLSCLIGYCSSHWCCKKEVQETRRERRRLMSMEMD

SEQ ID NO: 124 - protein, 56 aa, synthetic construct
DVLNAFKYPLLIGIGLSAVISLLSCLIGYCSSHWCCKKEVQETRRERRRLMSMEMD

SEQ ID NO: 125 - protein, 56 aa, synthetic construct
DVLNAFKYPLLIGIGLSAVITLLSCLIGYCSSHWCCKKEVQETRRERRRLMSMEMD

SEQ ID NO: 126 - protein, 56 aa, synthetic construct
DVLNAFKYPLLIGIGLSAVIGLLSCLISYCSSHWCCKKEVQETRRERRRLMSMEMD

SEQ ID NO: 127 - protein, 56 aa, synthetic construct
DVLNAFKYPLLIGIGLASVIGLLSCLIGYCSSHWCCKKEVQETRRERRRLMSMEMD

SEQ ID NO: 128 - protein, 56 aa, synthetic construct
DVLNAFKYPLLIGIGLSAVGILLSCLIGYCSSHWCCKKEVQETRRERRRLMSMEMD

SEQ ID NO: 129 - protein, 35 aa, synthetic construct
DVLNAFKYPLLIGVGLSTVIGLLSCLIGYCSSHWC

SEQ ID NO: 130 - protein, 35 aa, synthetic construct
DVLNAFKYPLLIGIGLSTVIGLLSCLIGYCSSHWC

SEQ ID NO: 131 - protein, 35 aa, synthetic construct
DVLNAFKYPLLIGVGLSAVIGLLSCLIGYCSSHWC

SEQ ID NO: 132 - protein, 35 aa, synthetic construct
DVLNAFKYPLLIGIGLSAVIGLLSCLIGYCSSHWC

SEQ ID NO: 133 - protein, 35 aa, synthetic construct
DVLNAFKYPLLIGILLSAVIGLLSCLIGYCSSHWC

SEQ ID NO: 134 - protein, 35 aa, synthetic construct
DVLNAFKYPLLIGIGLSLVIGLLSCLIGYCSSHWC

SEQ ID NO: 135 - protein, 35 aa, synthetic construct
DVLNAFKYPLLIGIGLSAVIGLLLCLIGYCSSHWC

SEQ ID NO: 136 - protein, 35 aa, synthetic construct
DVLNAFKYPLLIGIGLSAVIGLLSLLIGYCSSHWC

SEQ ID NO: 137 - protein, 35 aa, synthetic construct
DVLNAFKYPLLIGIGLSAVIALLSCLIGYCSSHWC

SEQ ID NO: 138 - protein, 35 aa, synthetic construct
DVLNAFKYPLLIGIGLSAVISLLSCLIGYCSSHWC

SEQ ID NO: 139 - protein, 35 aa, synthetic construct
DVLNAFKYPLLIGIGLSAVITLLSCLIGYCSSHWC

SEQ ID NO: 140 - protein, 35 aa, synthetic construct
DVLNAFKYPLLIGIGLSAVIGLLSCLISYCSSHWC

SEQ ID NO: 141 - protein, 35 aa, synthetic construct
DVLNAFKYPLLIGIGLASVIGLLSCLIGYCSSHWC

SEQ ID NO: 142 - protein, 35 aa, synthetic construct
DVLNAFKYPLLIGIGLSAVGILLSCLIGYCSSHWC

SEQ ID NO: 143 - protein, 1001 aa, synthetic construct
METDTLLLWVLLLWVPGSTGDGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPEFRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 144 - protein, 169 aa, synthetic construct
METDTLLLWVLLLWVPGSTGDGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPEFKYPLLIGVGLSTVIGLLSCLIGYCSS

SEQ ID NO: 145 - protein, 169 aa, synthetic construct
METDTLLLWVLLLWVPGSTGDGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPEFKYPLLIGVGLSAVIGLLSCLIGYCSS

SEQ ID NO: 146 - protein, 169 aa, synthetic construct
METDTLLLWVLLLWVPGSTGDGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPEFKYPLLIGIGLSTVIGLLSCLIGYCSS

SEQ ID NO: 147 - protein, 169 aa, synthetic construct
METDTLLLWVLLLWVPGSTGDGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPEFKYPLLIGIGLSAVIGLLSCLIGYCSS

SEQ ID NO: 148 - protein, 218 aa, synthetic construct
METDTLLLWVLLLWVPGSTGDYPYDVPDYAGAQPARSMEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPVDEQKLISEEDLNAVGQDTQEVIVVPHSLPFKVVVISAILALVVLTIISLIILIMLWQKKPR

SEQ ID NO: 149 - protein, 169 aa, synthetic construct
MMLQHLVIFCLGLVVQNFCSPGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPEFKYPLLIGIGLSAVIGLLSCLIGYCSS

SEQ ID NO: 150 - protein, 103 aa, synthetic construct
MLLTLIILLPVVSKFSFVSLSANSDSECPLSHDGYCLHDGVCMYIEALDKYACNCVVGYIGERCQYRDLKWWELREFKYPLLIGVGLSTVIGLLSCLIGYCSS

SEQ ID NO: 151 - protein, 103 aa, synthetic construct
MLLTLIILLPVVSKFSFVSLSANSDSECPLSHDGYCLHDGVCMYIEALDKYACNCVVGYIGERCQYRDLKWWELREFKYPLLIGVGLSAVIGLLSCLIGYCSS

SEQ ID NO: 152 - protein, 103 aa, synthetic construct
MLLTLIILLPVVSKFSFVSLSANSDSECPLSHDGYCLHDGVCMYIEALDKYACNCVVGYIGERCQYRDLKWWELREFKYPLLIGIGLSTVIGLLSCLIGYCSS

SEQ ID NO: 153 - protein, 103 aa, synthetic construct
MLLTLIILLPVVSKFSFVSLSANSDSECPLSHDGYCLHDGVCMYIEALDKYACNCVVGYIGERCQYRDLKWWELREFKYPLLIGIGLSAVIGLLSCLIGYCSS

SEQ ID NO: 154 - protein, 250 aa, synthetic construct
MLLTLIILLPVVSKFSFVSLSANSDSECPLSHDGYCLHDGVCMYIEALDKYACNCVVGYIGERCQYRDLKWWELRVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 155 - protein, 133 aa, synthetic construct
MLLTLIILLPVVSKFSFVSLSANSDSECPLSHDGYCLHDGVCMYIEALDKYACNCVVGYIGERCQYRDLKWWELREFDVLNAFKYPLLIGIGLSTVIGLLSCLIGYCSSHWCCKKEVQETRRERRRLMSMEMD

SEQ ID NO: 156 - protein, 112 aa, synthetic construct
MLLTLIILLPVVSKFSFVSLSANSDSECPLSHDGYCLHDGVCMYIEALDKYACNCVVGYIGERCQYRDLKWWELREFDVLNAFKYPLLIGIGLSTVIGLLSCLIGYCSSHWC

SEQ ID NO: 157 - protein, 129 aa, synthetic construct
MLLTLIILLPVVSKFSFVSLSANSDSECPLSHDGYCLHDGVCMYIEALDKYACNCVVGYIGERCQYRDLKWWELRVIVVAVCVVVLVMLLLLSLWGAHYYRTQEFHWCCKKEVQETRRERRRLMSMEMD

SEQ ID NO: 158 - protein, 178 aa, synthetic construct
METDTLLLWVLLLWVPGSTGDGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPEFDVLNAFKYPLLIGIGLSAVIGLLSCLIGYCSSHWC